(19) United States
(12) Patent Application Publication
(10) Pub. No.: US 2025/0174305 A1
(43) Pub. Date: May 29, 2025

(54) UTILIZING BIOLOGICAL MACHINE LEARNING REPRESENTATIONS AND A LANGUAGE MACHINE LEARNING MODEL FOR INITIATING COMPOUND EXPLORATION PROGRAMS

(71) Applicant: Recursion Pharmaceuticals, Inc., Salt Lake City, UT (US)

(72) Inventors: Brittney Mae Vierra, Salt Lake City, UT (US); Hayley Jeton Donnella, Austin, TX (US); Marta Marie Fay, Salt Lake City, UT (US); Michael Frank Cuccarese, Salt Lake City, UT (US); Vijay Shahani, Burlington (CA); Vivek Jayan, Dublin, CA (US); Conor Austin Forsman Tillinghast, Salt Lake City, UT (US); Safiye Celik, Sudbury, MA (US); Abraham Weintraub, Salt Lake City, UT (US); Sarah Karbalaei Khani, Coquitlam (CA); Gabriela Andrejeva, Sandy, UT (US)

(21) Appl. No.: 18/521,910

(22) Filed: Nov. 28, 2023

(51) Int. Cl. G16B 40/20; G06F 40/40; G06V 10/74; G06V 20/69; G16B 15/30; G16H 15/00; G16H 50/70

(52) U.S. Cl. CPC G16B 40/20; G06F 40/40; G06V 10/761; G06V 20/695; G06V 20/698; G16B 15/30; G16H 15/00; G16H 50/70

(57) ABSTRACT

The present disclosure relates to systems, non-transitory computer-readable media, and methods that implement a compound exploration initiation system. Indeed, in one or more implementations, the disclosed systems identify, from processed biological representations, a predicted biological relationship for an anchor compound or an anchor gene. Further, in one or more implementations, the disclosed systems generate digital text prompts that include text rating instructions for a language machine learning model from the predicted biological relationship. To illustrate, the disclosed systems generate rating metrics according to the text rating instructions utilizing the language machine learning model and combines the rating metrics to generate a program rating for the anchor gene or the anchor compound for initiating one or more compound exploration programs.

### BACKGROUND

Recent years have seen significant developments in hardware and software platforms for managing and operating complex computer-implemented pipelines. For example, conventional systems often utilize a variety of computing devices to attempt to validate and/or perform various tasks within a complex operational pipeline, such as compound exploration program. Such conventional systems, however, often utilize large computational data volumes, causing significant technical problems in validating compound program exploration tasks across computer devices and networks. Accordingly, conventional systems suffer from a number of technical deficiencies, particularly with regard to inaccuracy, inefficiency, and operational inflexibility of implementing computing devices.

### SUMMARY

Embodiments of the present disclosure provide benefits and/or solve one or more of the foregoing or other problems in the art with systems, non-transitory computer-readable media, and methods for utilizing processed biological representations and language machine learning models for initiating compound exploration programs. For example, the disclosed systems implement a language machine learning model to orchestrate a series of workflows to analyze genes and/or compounds for future exploration. In particular, in some embodiments, the disclosed systems identify predicted biological relationships for an anchor compound or an anchor gene from a processed biological representation (e.g., phenomic image embeddings or protein binding machine learning predictions). Moreover, in some embodiments, the disclosed systems generate digital text prompts that contain the anchor compound or the anchor gene with text rating instructions for the language machine learning model. Furthermore, in some embodiments, from the digital text prompts, the disclosed systems use the language machine learning model to generate corresponding rating metrics according to the text rating instructions. Moreover, in some embodiments the disclosed systems combine the rating metrics to generate a program rating for the anchor compound or the anchor gene for initiating one or more compound exploration programs.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates a schematic diagram of a system environment in which a compound exploration initiation system can operate in accordance with one or more embodiments.

FIG. 2 illustrates an overview figure of the compound exploration initiation system generating a program rating for a anchor gene or anchor compound in accordance with one or more embodiments.

FIG. 3 illustrates an example diagram of the compound exploration initiation system generating phenomic image embeddings in accordance with one or more embodiments.

FIG. 4 illustrates generating machine learning binding representations in accordance with one or more embodiments.

FIG. 5 illustrates generating digital text prompts in accordance with one or more embodiments.

FIG. 6 illustrates generating rating metrics and a program rating from digital text prompts in accordance with one or more embodiments.

FIG. 7 illustrates generating rating metrics from a language machine learning model and additional data sources in accordance with one or more embodiments.

FIG. 8 illustrates utilizing digital text prompts with a language machine learning model to initiate compound exploration in accordance with one or more embodiments.

FIG. 9 illustrates utilizing digital text prompts with a language machine learning model based on phenomic image embeddings and binding representations to initiate compound exploration in accordance with one or more embodiments.

FIG. 10 illustrates an example graphical user interface of a client device portraying rating metrics and a program rating in accordance with one or more embodiments.

FIG. 11 illustrates an example series of acts for utilizing a language machine learning model to generate a program rating in accordance with one or more embodiments.

FIG. 12 illustrates a block diagram of a computing device for implementing one or more embodiments.

### DETAILED DESCRIPTION

This disclosure describes one or more embodiments of a compound exploration initiation system that utilizes processed biological representations and language machine learning models to generate rating metrics and program ratings for compound exploration programs across computer networks. For example, in some embodiments the compound discovery process involves multiple groups of computing devices individually querying and extracting information related to compound exploration programs. In one or more implementations, the compound exploration initiation system analyzes compound exploration programs at scale by utilizing a language machine learning model. For instance, in some embodiments, the compound exploration initiation system utilizes processed biological representations to identify various predicted biological relationships and feeds those relationships to a language machine learning model with text rating instructions. To illustrate, the compound exploration initiation system utilizes the language machine learning model and text prompts to generate rating metrics and program ratings for anchor genes and/or anchor compounds. Moreover, the compound exploration initiation system combines individual rating metrics returned from the language machine learning model to generate an overall program rating. In some embodiments, the compound exploration initiation system intelligently decides whether to initiate one or more compound exploration programs utilizing the program rating.

As mentioned above, in one or more embodiments, the compound exploration initiation system utilizes processed biological representations to identify predicted biological relationships. In one or more embodiments, the compound exploration initiation system generates these machine learning representations based on various digital signals. For example, in some implementations, the compound exploration initiation system generates processed biological representations from phenomic digital images representing cell perturbations (e.g., gene knockouts or applying compounds to cells). Further, in some embodiments, the processed biological representations further include compound protein pocket interaction predictions. In some such embodiments, the processed biological representation includes machine learning binding representations that indicate the relationships between compounds and proteins. Moreover, in some embodiments the compound exploration initiation system obtains the processed biological representations from additional digital signals (e.g., multiomic datasets such as proteomics, metabolomics, invivomics, and transcriptomics) that contain information relating to genetic features, compound features, and/or protein features.

As mentioned above, in one or more implementations the compound exploration initiation system generates multiple digital text prompts based on predicted biological relationships. For example, the compound exploration initiation system stores digital text prompt templates within a digital text prompt repository and populates placeholder fields within the digital text prompt templates based on predicted biological relationships (e.g., an anchor compound or anchor gene) identified from processed biological representations. Moreover, in some embodiments the digital text prompts further include text rating instructions for the anchor compound or the anchor gene. Additionally, in some embodiments the digital text prompts also include context generation instructions.

As mentioned above, in some embodiments the compound exploration initiation system utilizes a language machine learning model to generate rating metrics according to text rating instructions of digital text prompts. For example, the compound exploration initiation system utilizes a large language model, transformer machine learning model, or other text-based machine learning architecture to process input digital text prompts having text rating instructions and generate rating metrics according to the text rating instructions. For example, the compound exploration initiation system can utilize a language machine learning model to generate gene impact rating metrics, previous analysis rating metrics, and/or tractability rating metrics from gene impact text prompts, previous analysis text prompts, and/or tractability text prompts, each having their own unique rating instructions (e.g., gene impact rating instructions, previous analysis rating instructions, and/or tractability rating instructions). In this manner, the compound exploration initiation system integrates a language machine learning model to generate precise and specific responses at scale (e.g., rating metrics on a full genomic scale). Further, in some embodiments, by utilizing the language machine learning model, the compound exploration initiation system is not restricted to a given dataset, but broadly peruses multiple datasets to generate rating metrics for a variety of different tasks in determining whether to initiate compound program exploration.

As mentioned above, in some embodiments the compound exploration initiation system dynamically combines rating metrics to generate a program rating. For example, the compound exploration initiation system utilizes the language machine learning model to generate rating metrics from digital text prompts, where the rating metrics include binary responses and/or scaled responses. For instance, in some embodiments the compound exploration initiation system takes the binary responses and/or scaled responses and combines them (e.g., via a combination algorithm) to generates a program rating. For instance, the compound exploration initiation system can utilize learned weights to combine individual rating metrics and generate an overall program rating for an anchor compound and/or anchor gene. Furthermore, in some embodiments in generating the program rating, the compound exploration initiation system determines whether a subset of rating metrics satisfies a predetermined rating metric threshold.

In addition, in one or more implementations, the compound exploration initiation system utilizes downstream analysis of compounds or genes to further learn and improve rating metrics and/or program ratings. For example, the compound exploration initiation system can monitor what compounds are selected for future hits or leads in compound discovery pipelines and then modify weights, combination algorithms, or other parameters to more accurately identify those biological relationships that will lead to successful compounds. Thus, the compound exploration initiation system can form a virtuous feedback loop to iteratively improve selected relationships to explore through additional programs.

As mentioned above, although conventional systems can validate and perform various tasks related to determining a biological relationship, such systems have a number of problems in relation to accuracy, efficiency, and flexibility of operation. For instance, conventional systems inaccurately explore some biological relationships due to the technical difficulties associated with examining and exploring disparate information stored across large data volumes. Indeed, conventional systems often fail to accurately filter through digital signals indicating millions of potential biological relationships to accurately focus on relationships that need additional computer-implemented analyses. To illustrate, conventional systems have access to various digital databases or other repositories having digital signals indicating potential relationships to explore. However, conventional systems cannot accurately extract and select the most promising relationships to explore from the potential millions (or billions) of pertinent combinations. Thus, conventional systems often inaccurately identify and select anchors/targets for initiating downstream compound analysis programs.

In addition to their inaccuracies, conventional systems are also inefficient. More specifically, conventional systems require an excessive number of interactions and graphical user interfaces to identify potential relationships from digital signals available across different computing devices or digital repositories. For instance, conventional systems require significant inputs and user interfaces to query, search, and review digital signals (e.g., digital articles, spreadsheets, or test results) and identify biological relationships for anchor compounds or anchor genes. The time, number of user interactions, and number of user interfaces required to search and review digital literature and datasets relating to potential biological relationships through conventional systems wastes significant computing resources (e.g., memory and processing power). Moreover, these inefficiencies become more and more pronounced as the number of desired relationships and the size/number of pertinent information sources increases.

Furthermore, in addition to their inaccuracies and inefficiencies, conventional systems suffer from operational inflexibility. Indeed, conventional systems cannot analyze digital databases effectively to filter and select pertinent anchors/targets for initiating compound exploration programs. Rather conventional systems rigidly rely on client device queries, including user interactions described above, to sort and analyze digital information to select compounds or genes to explore. Moreover, conventional systems lack the ability to scale to a large number (millions or billions) of potential biological relationships with regard to genes across the human genome, a litany of potential compounds, and various diseases/biological activities.

As suggested by the foregoing discussion, the compound exploration initiation system provides a variety of technical advantages relative to conventional systems. For example, by utilizing processed biological representations to generate digital text prompts for language machine learning models, the compound exploration initiation system accurately identifies and selects biological relationships for initiating compound exploration programs. For instance, in some embodiments the compound exploration initiation system identifies potential biological relationships from machine learning embeddings (e.g., processed biological representations such as image embeddings generated from cell perturbations or other machine learning predictions). Moreover, in some embodiments, the compound exploration initiation system further utilizes these potential biological relationships to generate dynamic text prompts for a language machine learning model. The language machine learning model thus determines rating metrics and corresponding program ratings to guide downstream computer-implemented processes. In this manner, the compound exploration initiation system can more accurately identify and select anchor genes and/or anchor compounds for initiating compound exploration programs.

Furthermore, in some embodiments, the compound exploration initiation system improves efficiency relative to conventional systems. Specifically, the compound exploration initiation system can identify a predicted biological relationship (e.g., from processed biological representations) and further generate digital text prompts to query the language machine learning model to return rating metrics. Thus, the compound exploration initiation system can significantly reduce interactions and interfaces required by conventional systems to search and review digital literature and datasets. Accordingly, the compound exploration initiation system can significantly reduce the time, number of user interactions, and number of user interfaces needed for comparing and analyzing potential biological relationships relative to conventional systems.

Moreover, by identifying predicted biological relationships, generating digital text prompts, and utilizing the language machine learning model, the compound exploration initiation system can improve operational flexibility relative to conventional systems. Specifically, by utilizing a language machine learning model to generate rating metrics, the compound exploration initiation system can intelligently analyze large repositories of data (e.g., with millions or billions of potential combinations of genes and/or compounds relative to biological activities/diseases) and validate potential biological relationships on a large scale for determining whether to initiate compound program exploration.

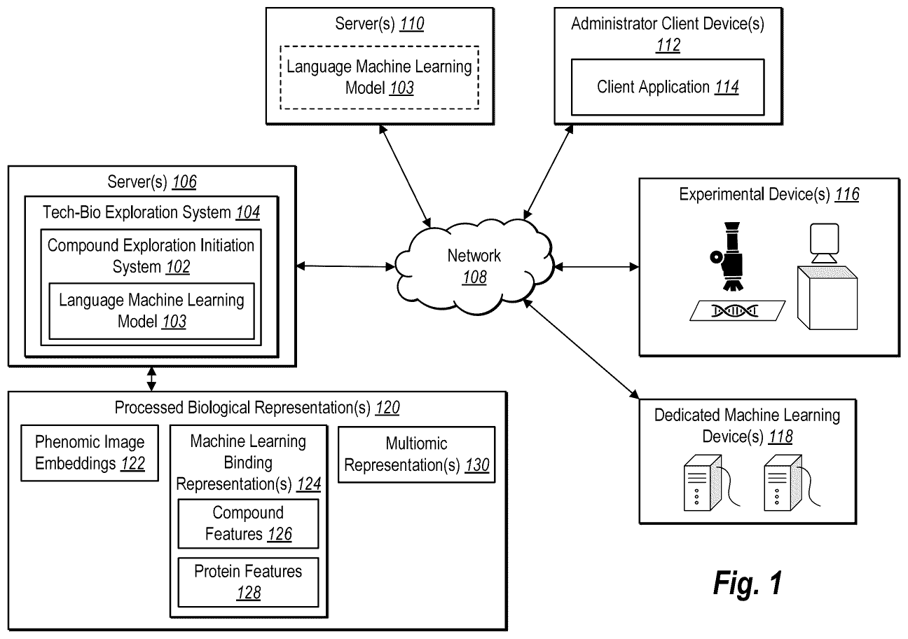

Additional detail regarding a compound exploration initiation system 102 will now be provided with reference to the figures. In particular, FIG. 1 illustrates a schematic diagram of a system environment in which the compound exploration initiation system 102 can operate in accordance with one or more embodiments.

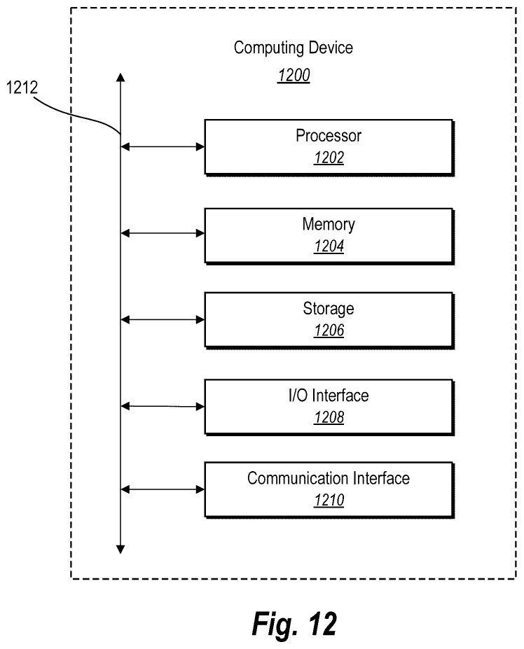

As shown in FIG. 1, the environment includes server(s) 106 (which includes a tech-bio exploration system 104, the compound exploration initiation system 102, and a language machine learning model 103), a network 108, server(s) 110, administrator client device(s) 112, experimental device(s) 116, dedicated machine learning device(s) 118, and various representations (e.g., processed biological representation(s) 120, machine learning binding representation(s) 124, and multiomic representation(s) 130). As further illustrated in FIG. 1, the various computing devices within the environment can communicate via the network 108. Although FIG. 1 illustrates the compound exploration initiation system 102 being implemented by a particular component and/or device within the environment, the compound exploration initiation system 102 can be implemented, in whole or in part, by other computing devices and/or components in the environment (e.g., the administrator client device(s) 112). Additional description regarding the illustrated computing devices is provided with respect to FIG. 12 below.

As shown in FIG. 1, the server(s) 106 (e.g., one or more local servers operated by a particular entity) can include the tech-bio exploration system 104. In some embodiments, the tech-bio exploration system 104 can determine, store, generate, and/or display tech-bio information including maps of biology, experiments from various sources, and/or machine learning tech-bio predictions. For instance, the tech-bio exploration system 104 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 104 provides an environment for operating, executing, and managing complex drug discovery pipelines.

For instance, the tech-bio exploration system 104 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 104 can generate or determine a variety of predictions and inter-relationships for improving treatments/interventions.

To illustrate, the tech-bio exploration system 104 can generate maps of biology indicating biological inter-relationships or similarities between these various input signals to discover potential new treatments as part of the complex compound discovery process. For example, the tech-bio exploration system 104 can utilize machine learning and/or maps of biology to identify a similarity between a first gene associated with disease treatment and a second gene previously unassociated with the disease based on a similarity in resulting phenotypes from gene knockout experiments. The tech-bio exploration system 104 can then identify new treatments based on the gene similarity (e.g., by targeting compounds the impact the second gene). Similarly, the tech-bio exploration system 104 can analyze signals from a variety of sources (e.g., protein interactions, or invivo experiments) to predict efficacious treatments based on various levels of biological data.

The tech-bio exploration system 104 can generate GUIs comprising dynamic user interface elements to convey tech-bio information and receive user input for intelligently exploring tech-bio information. Indeed, as mentioned above, the tech-bio exploration system 104 can generate GUIs displaying different maps of biology that intuitively and efficiently express complex interactions between different biological systems for identifying improved treatment solutions. Furthermore, the tech-bio exploration system 104 can also electronically communicate tech-bio information between various computing devices.

As shown in FIG. 1, the tech-bio exploration system 104 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 104 collects, manages, and transmits data across a variety of different entities, accounts, and devices. In some cases, the tech-bio exploration system 104 is a network system that facilitates access to (and analysis of) tech-bio information within a centralized operating system. Indeed, the tech-bio exploration system 104 can link data from different network-based research institutions to generate and analyze maps of biology.

As shown in FIG. 1, the tech-bio exploration system 104 can include a system that comprises the compound exploration initiation system 102 that identifies a predicted biological relationship, generates digital text prompts, generates rating metrics, and combines rating metrics to generate a program rating for initiating compound exploration programs. For example, in context of the above description for the tech-bio exploration system 104, in some embodiments the tech-bio exploration system 104 further utilizes the compound exploration initiation system 102 to enhance the validation and operation of dealing with a large volume of data for compound exploration programs (e.g., intelligently filtering a large number of potential biological relationships utilizing machine learning analysis down to a feasible number of potential relationships). For instance, the compound exploration initiation system 102 works in tandem with the tech-bio exploration system 104 to utilize a language machine learning model to generate rating metrics for an anchor compound or an anchor gene according to text rating instructions.

For example, as shown in FIG. 1, the compound exploration initiation system 102 integrates the language machine learning model 103 to generate rating metrics and program ratings for an anchor gene or anchor compound. Moreover, as further shown, in some embodiments, the compound exploration initiation system 102 utilizes the language machine learning model 103 from the server(s) 110 (e.g., a third-party server separate from the server(s) 106.).

As also illustrated in FIG. 1, the environment includes the administrator client device(s) 112. In some embodiments, the administrator client device(s) 112 can transmit a query corresponding to an anchor gene and/or anchor compound. In response, the administrator client device(s) 112 can receive rating metrics and program ratings for an anchor compound or anchor gene from the compound exploration initiation system 102 (e.g., via the language machine learning model 103) to determine whether to initiate compound program exploration. Thus, for example, the administrator client device(s) 112 can coordinate and orchestrate compound program exploration based on results received from the compound exploration initiation system 102. Specifically, the administrator client device(s) 112 via a client application 114 receives data from the compound exploration initiation system 102 and determines for example, to initiate additional machine learning analysis on certain predicted biological relationships and/or initiates compound program exploration programs. Moreover, in some embodiments the administrator client device(s) 112 includes multiple client devices that receive data from the compound exploration initiation system 102 and execute downstream machine learning analysis or initiate one or more compound exploration programs.

To illustrate, the administrator client device(s) 112 can include computing devices that implement, manage, or initiate a compound program exploration. For example, the administrator client device(s) 112 can receive data from the compound exploration initiation system 102 regarding an anchor gene or anchor compound and in response, the administrator client device(s) 112 can automatically generate additional machine learning representations, perform additional analysis, and/or initiate various compound exploration programs. In some embodiments, the administrator client device(s) 112 via the client application 114 (upon execution) cause the experimental device(s) 116 to perform various actions. Accordingly, a user can interact with the client application of the administrator client device(s) 112 to cause the experimental device(s) 116 to perform analyses, access results or perform other actions.

For example, a user of a user account can interact with the client application 114 on the administrator client device(s) 112 to execute experiments or other multi-faceted processes and to further access tech-bio information, initiate a request for validating gene/compound relationships, and/or accessing various data related to various processed biological representations.

As just mentioned, the environment includes the experimental device(s) 116. For example, the compound exploration initiation system 102 can utilize the experimental device(s) 116 to for example, generate cell perturbations, apply compounds to specific gene anchors, and/or perform gene target knockouts. For example, the tech-bio exploration system 104 can interact with the experimental device(s) 116 that include intelligent robotic devices and camera devices for generating and capturing digital images of cellular phenotypes resulting from different perturbations (e.g., genetic knockouts or compound treatments of stem cells). Similarly, the experimental device(s) 116 can include camera devices and/or other sensors (e.g., heat or motion sensors) capturing real-time information from animals as part of invivo experimentation. The tech-bio exploration system 104 can also interact with a variety of other experimental device(s) such as devices for determining, generating, or extracting gene sequences or protein information.

For example, the experimental device(s) 116 may include computing devices linked to biosensors, electrophysiological platforms, x-ray crystallography machines, liquid chromatography mass spectrometry systems, nuclear magnetic resonance spectrometers, mass spectrometers. In some implementations, the compound exploration initiation system 102 manages, schedules, executes, and tracks operation of the experimental device(s) 116 based on other events within the environment.

As further shown in FIG. 1, the environment includes the network 108. As mentioned above, the network 108 can enable communication between components of the environment. In one or more embodiments, the network 108 may include a suitable network and may communicate using a various number of communication platforms and technologies suitable for transmitting data and/or communication signals, examples of which are described with reference to FIG. 12. Furthermore, although FIG. 1 illustrates computing devices communicating via the network 108, the various components of the environment can communicate and/or interact via other methods (e.g., communicate directly).

In addition, the environment can also include dedicated machine learning device(s) 118. For example, the dedicated machine learning device(s) 118 can include computing devices or virtual machines dedicated to training or implementing large-scale machine learning models. For example, the dedicated machine learning device(s) 118 can generate machine learning predictions and/or embeddings based on digital biological data (e.g., digital images of phenotypes resulting from different perturbations or compound-protein interactions from compound features). For instance, as shown, in some embodiments the dedicated machine learning device(s) 118 generate phenomic image embedding(s) 122, compound features 126 and protein features 128 (e.g., part of the machine learning binding representation(s) 124), and the multiomic representation(s) 130.

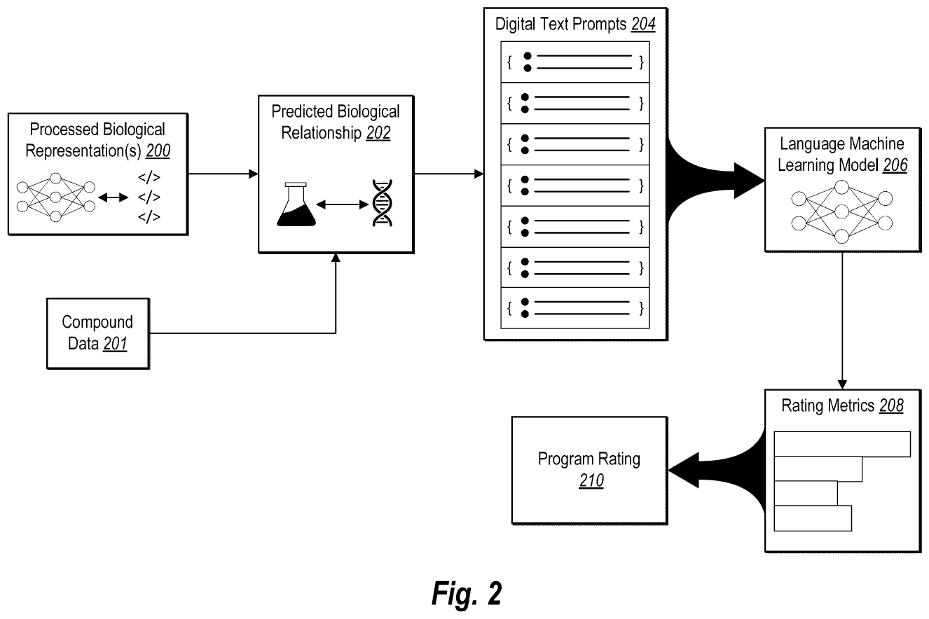

As mentioned above, in one or more implementations, the compound exploration initiation system 102 utilizes a language machine learning model to generate rating metrics and a program rating for initiating one or more compound exploration programs. For example, FIG. 2 shows an overview of the compound exploration initiation system 102 generating digital text prompts from a predicted biological relationships and subsequently a program rating in accordance with one or more embodiments.

As shown in FIG. 2, the compound exploration initiation system 102 utilizes processed biological representation(s) 200. In one or more embodiments, the compound exploration initiation system 102 identifies a predicted biological relationship 202 for an anchor compound or an anchor gene from the processed biological representation(s) 200. For example, for the processed biological representation(s) 200, the compound exploration initiation system 102 generates (e.g., via cell perturbations such as gene knockouts or applying a compound to a cell) embedding representations (e.g., a biological machine learning representation). For instance, the compound exploration initiation system 102 encodes or embeds digital images of the biological data utilizing a machine learning model. Accordingly, the embedding or encoding of biological data into the processed biological representation(s) 200 allows for the compound exploration initiation system 102 to identify variations relationships (e.g., not identifiable by human or manual means). Thus, for example, the processed biological representation(s) 200 can include representations from phenomic digital images, protein binding representations, invivomic representations, proteomic representations, or other information generated utilizing machine learning models. In other words, the processed biological representation(s) 200 includes biological machine learning representations (e.g., representations generated from machine learning models and/or machine learning embeddings).

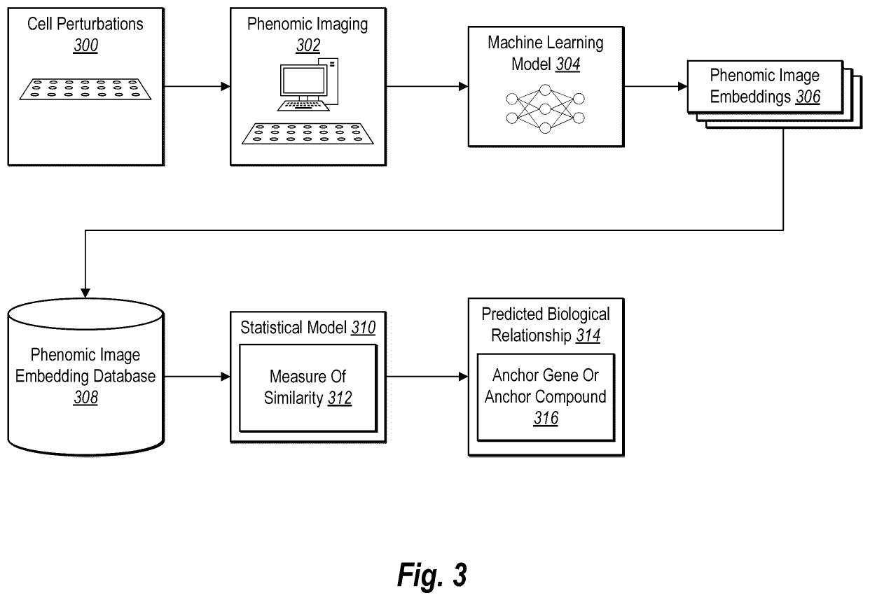

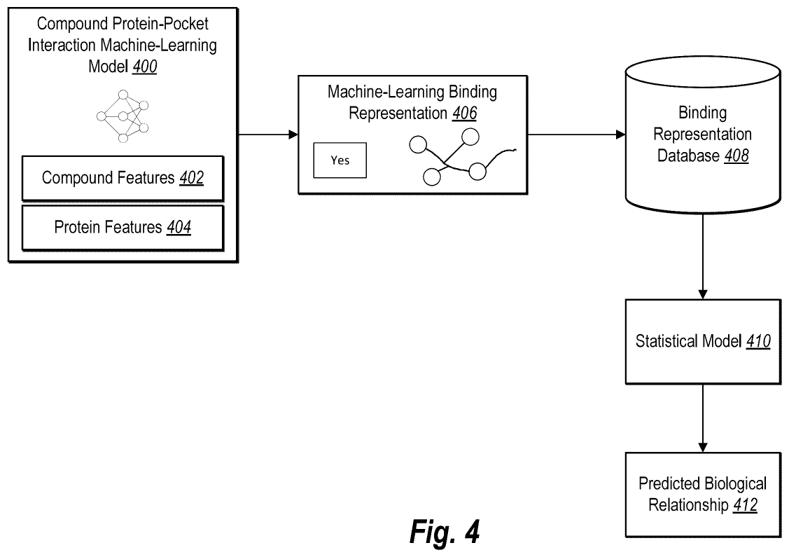

Moreover, the processed biological representation(s) further include Trekseq data, which includes RNA sequencing to determine the number of express proteins that map to a particular gene (e.g., knocking out a gene to see how much of a gene is expressed). Further, in some embodiments Trekseq involves analyzing the transcriptome of a cell, where transcriptome includes messenger RNA, non-coding RNA and other RNA molecules in a cell. Additional details regarding the processed biological representation(s) 200 is given below in the description of FIGS. 3 and 4.

Furthermore, as shown in FIG. 2, the compound exploration initiation system 102 further utilizes compound data 201. For example, the compound data 201 includes chemical data that represents potential novel chemistry (e.g., a novel chemical relationships) for which the compound exploration initiation system 102 uses as a starting point to further identify the predicted biological relationship 202 (e.g., utilizes potentially unique chemical signals to identify the predicted biological relationship 202). For instance, the compound exploration initiation system 102 utilizes the compound data 201 that originates from a binding representation database which is discussed in further detail below in FIG. 4. As shown in FIG. 2, the compound exploration initiation system 102 can utilize both the processed biological representation(s) 200 and the compound data 201 to identify the predicted biological relationship 202. In some instances, the compound exploration initiation system 102 utilizes one or the other to identify the predicted biological relationship 202.

As mentioned, from the processed biological representation(s) 200, the compound exploration initiation system 102 identifies the predicted biological relationship 202 for the anchor compound or the anchor gene. For example, the predicted biological relationship 202 can include a predicted biological connection or affiliation (e.g., corresponding to a gene or compound). For instance, a predicted biological relationship includes a hypothesis regarding an affiliation between a gene and/or compound relative to a disease, treatment, or biological activity. For example, a predicted biological relationship can include a predicted impact on a disease (e.g., cancer) relative to an anchor gene or anchor compound. Similarly, a predicted biological relationship can include a compound having a particular biological activity (that impacts a particular gene or protein). A predicted biological relationship can thus include relationships between genes, between compounds, between compounds and genes, and/or between diseases and compounds/genes.

In one or more embodiments an anchor gene includes a specific gene targeted or identified as part of a predicted biological relationship. To illustrate, an anchor gene can include a gene identified for a predicted function or activity (e.g., a predicted effect on a particular disease or condition). For instance, the compound exploration initiation system 102 identifies an anchor gene as part of the process of compound program exploration. Further, in some instances, the compound exploration initiation system 102 utilizes the anchor gene to identify compounds that interact with the anchor gene. Specifically, in the compound program exploration process, the compound exploration initiation system 102 utilizes compounds (e.g., anchor compounds) to inhibit or enhance the expression or function of an anchor gene.

In one or more embodiments, the compound exploration initiation system 102 identifies a predicted biological relationship for an anchor gene from a processed biological representation(s) (e.g., phenomic image embeddings or machine learning binding representations). Further, in some embodiments, the compound exploration initiation system 102 identifies the anchor gene from multiomic processes such as genomics, clinical genomics (e.g., measured genetic information from clinical treatment of humans with one or more biological conditions or diseases), transcriptomics, proteomics, and/or invivomics.

In one or more embodiments, an anchor compound includes a molecule (or soluble factor) targeted or identified as part of a predicted biological relationship. To illustrate, an anchor compound can include a molecule identified for a predicted function or activity (e.g., predicted to treat a particular disease or condition). For instance, in some embodiments the anchor compound has the potential to interact with a biological substrate such as a protein, enzyme, receptor, or gene that is associated with the particular disease or condition.

Similar to anchor genes, in some embodiments the compound exploration initiation system 102 identifies anchor compounds based on the processed biological representations 200 (e.g., phenomic image embeddings or machine learning binding representations). Furthermore, in some embodiments the compound exploration initiation system 102 identifies the anchor compound from identifying the anchor gene. For instance, from identifying a gene of interest that has a high correlation with a particular disease or condition, the compound exploration initiation system 102 further identifies an anchor compound with a statistically significant relationship with the anchor gene.

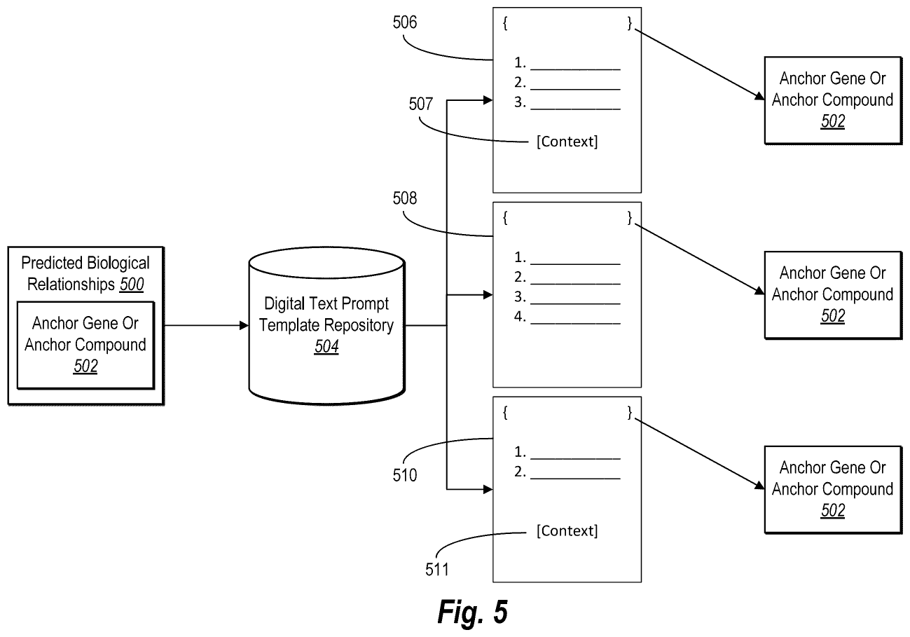

Further, as shown in FIG. 2, the compound exploration initiation system 102 utilizes the predicted biological relationships of an anchor compound or an anchor gene to generate digital text prompts 204. In one or more embodiments, a digital text prompt includes a prompt or query regarding a predicted biological relationship (e.g., for an anchor compound and/or an anchor gene). In particular, a digital text prompt includes a text query for submission to a language machine learning model. For instance, a digital text prompt includes a text query that references an anchor compound and/or an anchor gene (and additional instructions such as text rating instructions or context instructions). Moreover, the compound exploration initiation system 102 generates the digital text prompt based on the anchor compound and/or the anchor gene and provides the digital text prompt to a language machine learning model 206. Additional details regarding generating the digital text prompt are given below in the description of FIG. 5.

As mentioned, in one or more embodiments, the compound exploration initiation system 102 generates rating metrics utilizing the language machine learning model 206. As used herein, the term machine learning model includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques to improve in accuracy and/or effectiveness. Example machine learning models include various types of decision trees, support vector machines, Bayesian networks, random forest models, or neural networks (e.g., deep neural networks).

As used herein, a neural network includes a machine learning model of interconnected artificial neurons (e.g., organized in layers) that communicate and learn to approximate complex functions and generate outputs based on a plurality of inputs provided to the model. In some instances, a neural network includes an algorithm (or set of algorithms) that implements deep learning techniques that utilize a set of algorithms to model high-level abstractions in data. To illustrate, in some embodiments, a neural network includes a convolutional neural network, a recurrent neural network (e.g., a long short-term memory neural network), a transformer neural network, a generative adversarial neural network, a graph neural network, a diffusion neural network, or a multi-layer perceptron. In some embodiments, a neural network includes a combination of neural networks or neural network components.

As used herein, the term language machine learning model refers to a machine learning model that analyzes a language input (e.g., text or verbal input) to generate a predicted output. For instance, a language machine learning model includes a neural network that generates text based on an input text or query. The compound exploration initiation system 102 can utilize a variety of architectures for a language machine learning model, such as a large language model or other transformer neural network model. For instance, a large language model includes one or more neural networks capable of processing natural language text to generate outputs that range from predictive outputs, analyses, or combinations of data within stored content items. In particular, a large language model can include parameters trained (e.g., via deep learning) on large data volumes to learn patterns and rules of language for summarizing and/or generating digital content. Examples of large language model include BLOOM, Bard AI, ChatGPT (e.g., GPT-3, GPT-4, etc.), LaMDA, and/or DialoGPT. Moreover, in some embodiments a language transformer model includes bidirectional encoder representations (BERT), Robustly optimized BERT (ROBERTa), and other text transformer models.

Moreover, as shown in FIG. 2, the compound exploration initiation system 102 utilizes the language machine learning model 206 to generate rating metrics 208 from the digital text prompts 204. Specifically, the compound exploration initiation system 102 provides the digital text prompts 204 (including text rating instructions) to the language machine learning model 206. In one or more embodiments, the compound exploration initiation system 102 generates the rating metrics 208 according to the text rating instructions. Thus, a rating metric includes a value, score, or classification measure (as generated by a language machine learning model based on text rating instructions). Accordingly, for text rating instructions with a scoring rubric from 0-5, the compound exploration initiation system 102 receives from the language machine learning model a rating from 0 to 5 as the rating metric. Similarly, for a binary text rating instruction, the compound exploration initiation system 102 generates a binary (e.g., I/O, or yes/no) rating metric.

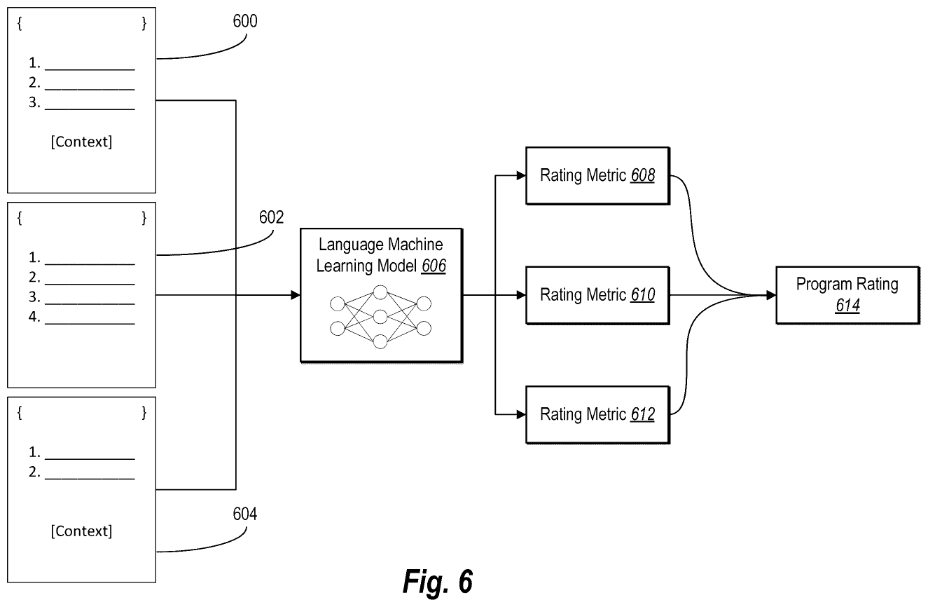

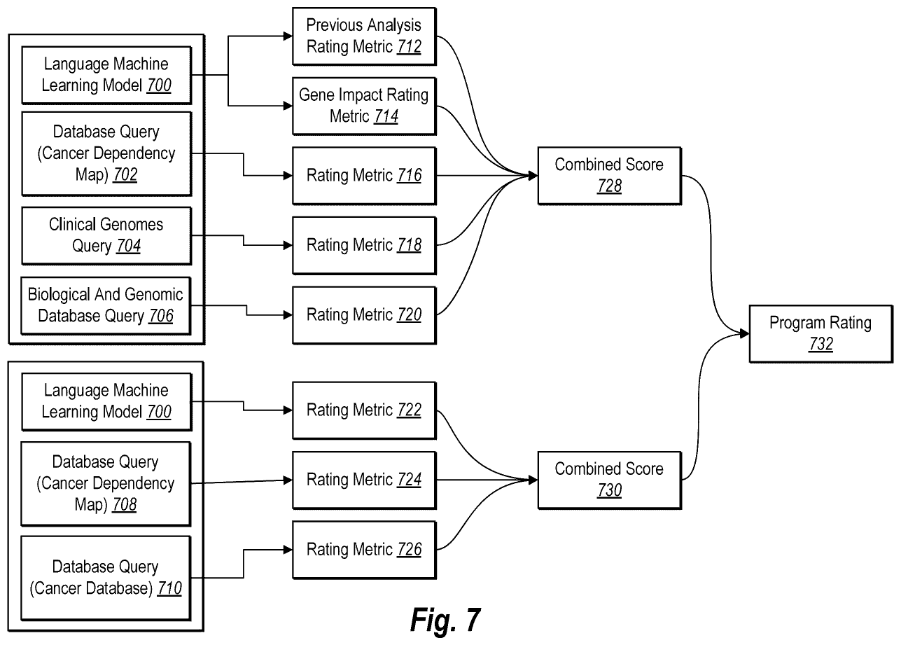

Furthermore, as shown, the compound exploration initiation system 102 combines the rating metrics 208 to generate the program rating 210. For instance, the compound exploration initiation system 102 receives the rating metrics 208 from the language machine learning model 206 and utilizes various weights of a computer-implemented model to combine the rating metrics 208 to determine the program rating 210. For example, in some embodiments the program rating 210 includes an average of the rating metrics 208 (e.g., a weighted average), a sum of the rating metrics 208, or a binary response. Specifically, the program rating 210 indicates whether to initiate one or more compound exploration programs. Additional details regarding the rating metrics 208 and the program rating 210 are given below in the description of FIGS. 6 and 7.

As mentioned above, the compound exploration initiation system 102 generates processed biological representations that include phenomic image embeddings. For example, FIG. 3 illustrates a diagram of the compound exploration initiation system 102 utilizing phenomic imaging to generate phenomic image embeddings in accordance with one or more embodiments.

As shown in FIG. 3, the compound exploration initiation system 102 performs cell perturbations 300. As used herein, the term “cell perturbation” (or simply perturbation) refers to an alteration or disruption to a cell or the cell's environment (to elicit potential phenotypic changes to the cell). In particular, the term perturbation can include a gene perturbation (i.e., a gene-knockout perturbation) or a compound perturbation (e.g., a molecule perturbation or a soluble factor perturbation). These perturbations are accomplished by performing a perturbation experiment. A perturbation experiment refers to a process for a perturbation to a cell. A perturbation experiment also includes a process for developing/growing the perturbed cell into a resulting phenotype.

Thus, a gene perturbation can include gene-knockout perturbations (performed through a gene knockout experiment). For instance, a gene perturbation includes a gene-knockout in which a gene (or set of genes) is inactivated or suppressed in the cell (e.g., by CRISPR-Cas9 editing).

Moreover, a compound perturbation can include a cell perturbation using a molecule and/or soluble factor. For instance, a compound perturbation can include reagent profiling such as applying a small molecule to a cell and/or adding soluble factors to the cell environment. Additionally, a compound perturbation can include a cell perturbation utilizing the compound or soluble factor at a specified concentration. Indeed, compound perturbations performed with differing concentrations of the same molecule/soluble factor can constitute separate compound perturbations. A soluble factor perturbation is a compound perturbation that includes modifying the extracellular environment of a cell to include or exclude one or more soluble factors. Additionally, soluble factor perturbations can include exposing cells to soluble factors for a specified duration wherein perturbations using the same soluble factors for differing durations can constitute separate compound perturbations.

Thus, for example, the compound exploration initiation system 102 performs the cell perturbations 300 by performing gene perturbations, compound perturbations or other perturbation processes. To illustrate, cell perturbations can include thawing cells, plating them, transfection (for CRISPR-treated wells), adding compounds or soluble factors, fixation, staining, and (ultimately) imaging. To illustrate, FIG. 3 shows the compound exploration initiation system 102 performing phenomic imaging 302 on the cell perturbations 300. For example, after inducing the cell perturbations, the compound exploration initiation system 102 utilizes a digital camera to capture digital images of the cell perturbations 300. For instance, the phenomic imaging 302 can include imaging multiple cells (within a well of a plate) with the same perturbation applied. Moreover, the phenomic imaging 302 includes capturing digital perturbation images across different wells (of one or more plates) that include cells with different perturbations. The compound exploration initiation system 102 can perturb different genes utilizing different compounds at different concentrations for different durations to generate different cell phenotypes and different phenomic digital images.

Furthermore, FIG. 3 shows the compound exploration initiation system 102 utilizing a machine learning model 304 to generate phenomic image embeddings 306. As mentioned above, the compound exploration initiation system 102 can utilize a variety of machine learning architectures for the machine learning model 304 in generating the phenomic image embeddings 306. For instance, in some embodiments, the compound exploration initiation system 102 utilizes a deep image embedding model (e.g., a neural network such as a convolutional neural network).

For example, upon capturing phenomic digital images, the compound exploration initiation system 102 utilizes a deep image embedding model to generate phenomic image embeddings. For instance, a deep image embedding model includes a neural network (e.g., a convolutional neural network) or other embedding model that generates a vector representation of an input digital image.

In some implementations, the compound exploration initiation system 102 trains the deep image embedding model through supervised learning (e.g., to predict perturbations from digital images). For instance, the compound exploration initiation system 102 trains the deep image embedding model to generate predicted perturbations from phenomic digital images. For instance, perturbation mapping system utilizes neural network layers to generate vector representations of the phenomic digital images at different levels of abstraction and then utilizes output layers to generate predicted perturbations. The compound exploration initiation system 102 then trains the deep image embedding model by comparing the predicted perturbations with ground truth perturbations. Although the foregoing example describes a particular training approach and embedding model, the compound exploration initiation system 102 can utilize a variety of image embedding models.

With regard to FIG. 3, the compound exploration initiation system 102 utilizes the machine learning model 304 to generate embeddings (e.g., feature/vector representations) of new phenomic digital images. For instance, the compound exploration initiation system 102 utilizes the internal neural network layers to generate embeddings (rather than generate perturbation predictions). The compound exploration initiation system 102 then utilizes the embeddings as representations of the phenomic digital images.

Thus, utilizing the convolutional neural network, the compound exploration initiation system 102 can embed each image into a low dimensional feature space (e.g., as a phenomic image embedding). Accordingly, a phenomic image embedding refers to a numerical feature representation (e.g., a feature vector) of a phenomic digital image generated by a machine learning model. Indeed, the compound exploration initiation system 102 can generate a multi-dimensional representation of each image within the low dimensional feature space. These multi-dimensional representations thus represent the features of different underlying perturbations (e.g., genes and compounds) as reflected in phenomic digital images utilized to generate the embeddings.

As mentioned above, in one or more embodiments, the compound exploration initiation system 102 utilizes the phenomic image embeddings 306 as the processed biological representations. The compound exploration initiation system 102 utilizes the phenomic image embeddings 306 to determine predicted biological relationship 314. For example, the compound exploration initiation system 102 compares the phenomic image embeddings 306 in an embedding feature space to determine the predicted biological relationship 314.

As illustrated, in some embodiments the compound exploration initiation system 102 stores the phenomic image embeddings 306 in a processed biological representation database. Specifically, as shown in FIG. 3, the compound exploration initiation system 102 stores the phenomic image embeddings 306 in a phenomic image embedding database 308 for retrieval and analysis. For instance, the compound exploration initiation system 102 can receive a query regarding various genes and/or compounds, access the phenomic image embeddings 306 from the phenomic image embedding database 308, and generate the predicted biological relationship 314 (e.g., to provide a response to the query in real time).

Furthermore, as shown, in one or more implementations the compound exploration initiation system 102 utilizes a statistical model 310 to compare embeddings of the phenomic image embedding database 308 to identify potential biological relationships. Specifically, the statistical model 310 includes determining a measure of similarity 312 between phenomic image embeddings 306. For instance, the measure of similarity 312 includes determining cosine similarity between the phenomic image embeddings 306. The measure of similarity 312 can also include a distance measure (e.g., Euclidean distance) within an embedding feature space.

The compound exploration initiation system 102 can utilize a threshold similarity to compare the phenomic image embeddings 306 (e.g., those embeddings satisfying the threshold similarity are identified for the predicted biological relationship 314). Further, in some embodiments the compound exploration initiation system 102 utilizes a significance threshold (e.g., a statistical threshold). To illustrate, the compound exploration initiation system 102 can create a statistical distribution of cosine similarity scores for the phenomic image embeddings 306 (e.g., for a specific gene or compound). The compound exploration initiation system can utilize a significance threshold (e.g., p-value of 0.01) to determine the predicted biological relationship 314.

As shown in FIG. 3, based on the statistical model 310, the compound exploration initiation system 102 utilizes the measure of similarity 312 determined for the phenomic image embeddings 306 to identify a predicted biological relationship 314. For instance, the compound exploration initiation system 102 identifies an anchor gene or anchor compound 316 having a threshold similarity (at a threshold significance) relative to a gene having a known biological function. Thus, for example, the compound exploration initiation system 102 identifies a first gene that is known to have a function in cancer development. The compound exploration initiation system 102 determines a measure of similarity between the first gene and the anchor gene or anchor compound 316. If the measure of similarity satisfies a threshold, the compound exploration initiation system 102 determines the predicted biological relationship 314; namely, that the anchor gene or anchor compound 316 also has an impact on cancer development.

In some implementations, the compound exploration initiation system 102 determines the predicted biological relationship 314 based on user interaction with a graphical user interface element illustrating similarity measures between genes and/or compounds. For example, the compound exploration initiation system 102 can provide a user interface that includes a table or heatmap of similarity measures between genes and compounds. In response to selection of a particular field of the table or heatmap (e.g., a field showing a similarity measure), the compound exploration initiation system 102 can select the predicted biological relationship 314.

Although FIG. 3 illustrates generating the predicted biological relationship 314 based on the phenomic image embeddings 306, the compound exploration initiation system 102 can utilize other processed biological representations to determine predicted biological relationships. For instance, as mentioned, the compound exploration initiation system 102 generates machine learning binding representations and further utilizes the machine learning binding representations to identify predicted biological relationships. For example, FIG. 4 illustrates the compound exploration initiation system 102 utilizing a machine learning model to generate machine learning binding representations to store within a database in accordance with one or more embodiments.

As shown in FIG. 4, the compound exploration initiation system 102 utilizes a compound protein-pocket interaction machine-learning model 400. In some embodiments, the compound protein-pocket interaction machine-learning model 400 comprises a classification machine learning model trained to determine whether a compound will bind to a particular protein pocket (e.g., binding site). The compound protein-pocket interaction machine-learning model 400 can utilize a variety of compound features 402 (properties or characteristics of a compound) and/or protein features 404 (properties or characteristics of a protein) in generating a prediction. For instance, the compound exploration initiation system 102 can determine local protein features, global protein features, protein functional features, and/or compound/ligand fingerprints and analyze these features to generate the prediction.

As shown in FIG. 4, based on the compound features 402 and the protein features 404, the compound exploration initiation system 102 utilizes the compound protein-pocket interaction machine-learning model 400 to generate a machine-learning binding representation 406. In some embodiments the compound exploration initiation system 102 trains the compound protein-pocket interaction machine-learning model 400 to generate binary predictions for binding sites. In some embodiments the compound exploration initiation system 102 then strips one or more layers from the trained compound protein-pocket interaction machine-learning model to determine a match score (e.g., binding likelihood indicating a likelihood that the compound will bind at the corresponding protein pocket) for compound protein-pocket pairs. In some implementations, the match score can include a binary score (e.g., indicating that a compound will or will not bind at the binding site).

The compound exploration initiation system 102 can utilize the machine-learning binding representation 406 to determine a predicted biological relationship 412. For instance, the compound exploration initiation system 102 can utilize the machine-learning binding representation to identify proteins (and/or related genes) that will be impacted by a particular molecule. For example, consider a particular gene known to have a particular function (e.g., cancer impact). The compound exploration initiation system 102 can also identify a particular protein resulting from the particular gene (e.g., the protein resulting in a cell from transcribing the gene). The compound exploration initiation system 102 can utilize the compound protein-pocket interaction machine-learning model 400 to generate a machine learning binding representation 406 for a compound relative to the particular protein (e.g., a prediction of whether the compound will bind to a protein pocket of the particular protein). The compound exploration initiation system 102 can then generate the predicted biological relationship 412 based on the machine learning binding representation 406. For example, if the machine-learning binding representation 406 indicates that the compound will bind to the particular protein, the machine learning binding representation 406 can generate the predicted biological relationship 412; namely, that the compound may impact the function (e.g., cancer) correlated to the particular gene that results in the particular protein.

As shown in FIG. 4, in some implementations, the compound exploration initiation system 102 stores the machine-learning binding representation 406 in a binding representation database 408 (e.g., for storage and retrieval in responding to a query from a client device). From the binding representation database 408, the compound exploration initiation system 102 can utilize a statistical model 410 to identify the predicted biological relationship 412. For instance, like the description above in FIG. 3, the compound exploration initiation system 102 can utilize a statistical significance threshold with the statistical model to identify the predicted biological relationships 412. To illustrate, the compound exploration initiation system 102 identifies the predicted biological relationship 412 based on a prediction that satisfies a statistical significance threshold (e.g., a statistical p-value below a p-value threshold). To illustrate, the predicted biological relationship 412 can include a relationship between a compound and a protein, a relationship between a compound and a gene related to the protein, and/or a relationship between a compound and a particular biological activity or disease.

Although not shown in FIG. 4, in one or more embodiments, the compound exploration initiation system 102 identifies the predicted biological relationship 412 in an algorithm or process as follows:
1) for predicted anchors (e.g., anchor genes or anchor compounds), the statistical model identifies anchors that have a tractability greater than a certain predetermined threshold (e.g., 0.5). To illustrate, the compound exploration initiation system 102 can use statistical tests to determine if the expression of an anchor gene (e.g., for a protein) is significantly different between normal and diseased states, where a p-value for tractability can indicate a significance of the difference in expression levels. For instance, for a predicted anchor, the compound exploration initiation system 102 assesses statistical biological relevance (e.g., from the binding representation database 408) to a disease or condition of interest. Further, in some such instances, this can include assessing (e.g., from the binding representation database 408) characteristics of the predicted anchor being amenable to modulation by certain compounds.
2) Identify drug anchor interactions (e.g., a molecule or a specific protein involved in a disease process, utilized as an anchor for a drug to interact with), for further reducing the tractability list obtained from step 1. For example, the compound exploration initiation system can filter down the tractability list to satisfy a threshold of a predetermined number (e.g., filter the tractability list down to two hundred). For instance, based on the existing literature, and/or the binding representation database 408, obtain information related to molecular docking, laboratory assays, and cell based assays. Further, based on the obtained information indicating statistically significant drug target interactions (e.g., amongst the tractability list), reduce the tractability list down to only include drug target interactions that satisfy a statistical threshold.
3) Identify from the second step, anchors with a pocket confidence (e.g., predicting ligand binding sites on proteins) less than a predetermined threshold (e.g., confidence <0.05). For instance, based on step 2, the compound exploration initiation system 102 predicts a likelihood that a predicted protein (e.g., pocket) is a ligand-binding site. Accordingly, for a confidence of <0.05, this indicates a very high confidence of the pocket being a true ligand site. To illustrate, the compound exploration initiation system 102 uses the binding representation database 408 to predict ligand binding pockets on a protein of interest and a score for each pocket that indicates a level of confidence of the pocket being a ligand site.
4) Identify a minimum compound count (e.g., a number of unique chemical compounds that a drug discovery program aims to synthesize and test) to anchor proteins from the previous step. For instance, a minimum compound count includes identifying a number of compounds to be tested in an assay to identify potential lead compound for a compound discovery program.
5) From the previous step, map a number of compounds based on a statistical score (e.g., a p-value of significant binding affinity with a gene) to a statistical distribution. Further, from the statistical distribution, identify compounds without prediction skew (e.g., when a model has a bias to consistently make an incorrect prediction in a certain direction). For instance, the compound exploration initiation system 102 utilizes a statistical distribution chart to assess whether the data is right-skewed, left-skewed, or symmetrical.
6) Out of the identified compounds in step 5, further identify compounds without strong toxic signals (e.g., less than a predetermined threshold). For instance, the compound exploration initiation system 102 obtains from the binding representation database 408, and existing literature, compounds with less than predetermined number of toxic signals. To illustrate, toxic signal data can further be obtained from animal models, in vitro assays, in silico predictions, and high-throughput screenings.
7) Identify from the compounds in step 6, compounds with more than a predetermined number of Murko scaffolds (e.g., a simplified molecular structure derived by removing certain functional groups and leaving behind a core molecular structure). For instance, the compound exploration initiation system 102 identifies a number of Murko scaffolds by using a chemical compound dataset to extract Murcko scaffolds from compound structures.

Although the above description has a specific number of steps and order of steps, in one or more embodiments the compound exploration initiation system 102 omits, reorders, or adds one or more steps to identify a predicted biological relationship for an anchor gene or anchor compound.

In one or more embodiments, the compound protein-pocket interaction machine-learning model 400 can include a variety of machine learning model architectures. In some implementations, the compound protein-pocket interaction machine-learning model 400 includes supervised discriminative classifications or regression models such as a random forest, support vector machine, single layer perceptron, or multiple layer artificial neural network. In some embodiments, the compound protein-pocket interaction machine-learning model 400 takes the form of a fully-connected neural network with a feature input layer, and hidden layers with, and output nodes corresponding to interacting and non-interacting pairs. In some embodiments, an artificial neural network with multiple hidden layers omits connections between input types, for the creation of separate latent spaces representing ligand fingerprints, global protein features, local protein features, and protein functional features.

Further, in some embodiments the compound exploration initiation system 102 trains the compound protein-pocket interaction machine-learning model 400 by identifying a plurality of ghost ligands/compounds (and confidence scores) relative to particular proteins. In particular, the compound exploration initiation system 102 generates synthetic data by determining ghost compounds similar to selected compounds and proteins based on the confidence scores. The compound exploration initiation system 102 trains the compound protein-pocket interaction machine-learning model 400 based on features corresponding to known and synthetic compounds and proteins. For example, in one or more implementations, the compound exploration initiation system 102 trains and utilizes a compound protein interaction machine-learning model as described in METHOD AND SYSTEM FOR PREDICTING DRUG BINDING USING SYNTHETIC DATA, application Ser. No. 17/420,582, filed Jan. 2, 2020, which is incorporated by reference herein in its entirety.

Although FIGS. 3 and 4 illustrate the compound exploration initiation system 102 utilizing phenomic image embeddings and machine learning binding representations, in one or more embodiments, the compound exploration initiation system 102 also utilizes other machine learning representations and/or multiomic representations. For instance, the compound exploration initiation system 102 constructs other machine learning representations or databases that include information related to genomics, clinical genomics, invivomics, transcriptomics, proteomics, and metabolomics.

For example, in some embodiments genomics includes representations based on genes and inter-gene interactions, as well as a representation of the identification and characterization of the genetic makeup of a specific organism. Moreover, in some embodiments the compound exploration initiation system 102 utilizes a variety of bioinformatic tools to extract genes for a genome. Further, in some embodiments the clinical genomics includes representations based on an intersection between biological data and human health. For instance, clinical genomics includes determining genetics of a human organism (e.g., DNA) and/or RNA, mRNA, metabolites, proteins, and/or health records associated with a certain condition/disease.

To illustrate, invivomics includes representations, machine learning embeddings or predictions from in vivo data (e.g., experiments conducted in a living organism). Further, to illustrate, invivomic machine learning models can generate machine learning liability predictions or embeddings based on digital video and/or other digital signals from sensors of intelligent cages holding animals. Further, transcriptomics includes representations, machine learning predictions, or embeddings based on transcription mechanisms. Similarly, proteomics includes representations, machine learning predictions or embeddings that indicate protein information in a biological system and metabolomics includes representations, machine learning predictions, or embeddings that indicate metabolites in biological systems. The compound exploration initiation system 102 can utilize one or a combination of these various representations to generate digital text prompts, program ratings, and initiate compound exploration programs.

As mentioned above, the compound exploration initiation system 102 generates digital text prompts from a predicted biological relationship. For example, FIG. 5 illustrates the compound exploration initiation system 102 generating digital text prompts utilizing an anchor gene or anchor compound related to the predicted biological relationship in accordance with one or more embodiments.

As shown in FIG. 5, the compound exploration initiation system 102 generates or identifies a predicted biological relationship 500 that includes an anchor gene or anchor compound 502. The compound exploration initiation system 102 can determine the predicted biological relationship 500 utilizes a processed biological representation and/or one or more other processes described herein (e.g., as described in FIGS. 2-4). In some implementations, the compound exploration initiation system 102 identifies the predicted biological relationships 500 from user input at a client device (e.g., a client device query identifying the predicted biological relationships 500 and the anchor gene or anchor compound 502).

As shown in FIG. 5, the compound exploration initiation system 102 identifies a plurality of digital text prompt templates from a digital text prompt template repository 504. In one or more implementations, the compound exploration initiation system 102 generates the digital text prompt template repository 504 by combining a variety of digital text prompts corresponding to different digital text prompt types or classifications.

For instance, in some embodiments the compound exploration initiation system 102 generates the digital text prompt template repository 504 by receiving a plurality of digital text prompt templates from an administrator computing device. Further, in some embodiments the compound exploration initiation system 102 receives indications from the administrator computing device registering each of the plurality of digital text prompt templates with a specific type or classification.

Moreover, in one or more embodiments, the compound exploration initiation system 102 generates the digital text prompt template repository 504 by utilizing a generative model to generate a plurality of digital text prompt templates. In particular, the compound exploration initiation system 102 provides a request to generate specific digital text prompt types or classifications (e.g., to a trained prompt generation machine learning model). Further, in some embodiments the compound exploration initiation system 102 receives the plurality of digital text prompt templates back from the generative model and stores the plurality of digital text prompt templates in the digital text prompt template repository 504 tagged with a specific type or classification.

In one or more embodiments, the compound exploration initiation system 102 utilizes the digital text prompt template repository 504 to store multiple digital text prompt templates. For instance, the digital text prompt templates include pre-defined prompts to query a language machine learning model for biological information relating to a specific compound or gene. Moreover, in some embodiments the digital text prompt templates include pre-defined prompts with placeholder fields for inserting or populating the placeholder fields with an anchor gene or anchor compound.

As shown in FIG. 5, the compound exploration initiation system 102 selects one or digital text prompt templates from the digital text prompt template repository 504 based on the predicted biological relationships 500. For instance, as mentioned, the digital text prompt template repository 504 can contain a plurality of digital text prompt templates. Further, the compound exploration initiation system 102 can generate a mapping between various predicted biological relationships and the digital text prompt templates (e.g., the compound exploration initiation system 102 maps relationships between a particular biological query related to a disease, anchor compound, and/or or anchor gene to a particular digital text prompt type or classification). For example, the compound exploration initiation system 102 can determine that the predicted biological relationships 500 include the anchor gene or anchor compound 502 and a corresponding disease. The compound exploration initiation system 102 can filter or select those digital text prompts that relate to (e.g., have classifications or tags for) the specific disease, one or more characteristics of the anchor gene (e.g., templates corresponding to a particular gene cluster or function), and/or one or more characteristics of the anchor compound (e.g., templates corresponding to a particular class of compounds).

Moreover, in one or more embodiments, the compound exploration initiation system 102 utilizes an intelligent model to select one or digital text prompt templates from the digital text prompt template repository 504. For instance, in some such embodiments the compound exploration initiation system 102 utilizes a mapping machine learning model trained on various predicted biological relationships and digital text prompt templates (e.g., inputs to the model) to generate an output of a digital text prompt template (or a digital text prompt) most similar to a predicted biological relationship. Accordingly, in some cases the compound exploration initiation system 102 implements the mapping machine learning model to select the digital text prompts 506, 508, and 510.

To illustrate, in some embodiments the compound exploration initiation system 102 feeds as input the predicted biological relationships 500 into an encoder of the mapping machine learning model. In some such instances, the compound exploration initiation system 102 generates an embedding of the predicted biological relationships 500 and compares the embedding in a latent vector space to identify a similar embedding for digital text prompt templates. Based on the embedding comparison, the compound exploration initiation system 102 can generate an output of digital text prompt templates that satisfy a threshold with the predicted biological relationships 500.

Further, in one or more embodiments, the digital text prompt template repository 504 includes a plurality of different digital text prompt template types or classifications. For example, digital text prompt template types or classifications include previous analysis digital text prompt templates, gene impact digital text prompt templates, and tractability digital text prompt templates. Thus, in some embodiments the compound exploration initiation system 102 identifies the predicted biological relationships 500 for the anchor gene or anchor compound 502 and selects digital text prompt templates based on the above-mentioned types or classifications. Additional specific examples of each of these digital text prompt template types or classifications are given below in the description of FIGS. 7-9.

As shown in FIG. 5, the compound exploration initiation system 102 populates placeholder fields of the digital text prompts 506, 508, and 510 with the anchor gene or anchor compound 502 from the predicted biological relationship 500. The compound exploration initiation system 102 can also populate the digital text prompts 506, 508, 510 with other information from the predicted biological relationship 500. For example, the compound exploration initiation system 102 can populate placeholder fields for a particular biological activity or disease corresponding to the predicted biological relationship 500. Thus, for example, the compound exploration initiation system 102 can indicate that an anchor compound or anchor gene corresponds to a particular disease (e.g., cancer or diabetes). In some embodiments, the compound exploration initiation system 102 utilizes the pre-engineered prompts in context of the predicted biological relationship 500, to further validate the predicted relationship via a language machine learning model.

Moreover, as shown in FIG. 5, the compound exploration initiation system 102 generates digital text prompts 506, 508, and 510 from the digital text prompt template repository 504. In one or more embodiments, the digital text prompts 506, 508, and 510 include text rating instructions. For example, text rating instructions include a text description of a scoring rubric or classification (e.g., binary, class, or scaled). In particular, text rating instructions include text instructions to provide to the language machine learning model to output a score or classification based on different indications. In particular, a text rating instruction can include text providing a description of a particular scoring, rating, or classification approach. For instance, the text rating instructions can include text instructions for a scoring rubric from 0-5 (e.g., where 0 is the lowest and 5 is the highest) instructing the language machine learning model to rate a gene or compound according to the scoring rubric. Further, in some instances the text rating instructions include text describing a binary (e.g., I/O or “yes”/“no”) indication relating to a gene or compound.

Further, as shown in FIG. 5, the digital text prompts 506 and 510 further include context generation instructions 507 and 511. For example, the context generation instructions 507 and 511 includes (e.g., in addition to the text rating instructions) instructions directions for contextual information (e.g., to provide to a language machine learning model to output an explanation or an indication of information corresponding to an anchor gene, anchor compound, or rating metric). In particular, a context generation instruction can include text providing a description of contextual information regarding an anchor gene, anchor compound, and/or scoring metric. For instance, the context generation instructions 507 and 511 could read “return evidence linking {anchor gene or anchor compound} to cancer development and progression.” Further, in some embodiments the context generation instructions 507 and 511 could read “return the most important data points utilized to make the metric rating determination.” Additionally, in some embodiments, the context generation instructions 507 and 511 can include any other information variation related to returning information related to compound program exploration (e.g., number of estimated cases, deaths per year, number of studies already performed, specific relationships, and/or symptoms).

Although FIG. 5 shows a specific ordering of identifying digital text prompt templates from the digital text prompt template repository 504, in one or more embodiments, the compound exploration initiation system 102 does not utilize digital text prompt templates that include pre-defined prompts with placeholder fields. For instance, in some such embodiments the compound exploration initiation system 102 receives an indication of the predicted biological relationship 500, and directly generates the digital text prompts 506, 508, and 510 (e.g., by utilizing a trained prompt generation model to generate the digital text prompts 506, 508, and 510 for the predicted biological relationship 500).

Further, although FIG. 5 shows the digital text prompts 506 and 510 including the context generation instructions 507 and 511, in some embodiments, the compound exploration initiation system 102 identifies digital text prompt templates without any context generation instructions. Whereas in some embodiments, the compound exploration initiation system 102 only identifies digital text prompt templates with context generation instructions. In some embodiments, the compound exploration initiation system 102 can receive an indication from an administrator computing device whether to identify digital text prompt templates with context generation instructions.

Moreover, although FIG. 5 shows three digital text prompts (e.g., digital text prompts 506, 508, and 510) in some embodiments the compound exploration initiation system 102 can identify different numbers digital text prompts (e.g., hundreds or thousands) to utilize for the predicted biological relationships 500. Further, although FIG. 5 generally discusses the generation of digital text prompts, the description below provides specific examples of the compound exploration initiation system 102 utilizing prompts for gene impact, previous analysis, and tractability.

FIG. 6 illustrates a diagram of the compound exploration initiation system 102 utilizing a language machine learning model to generate rating metrics and a program rating in accordance with one or more embodiments. For example, FIG. 6 shows the compound exploration initiation system 102 passing digital text prompts 600-604 to a language machine learning model 606.

As just described in relation to FIG. 5, the digital text prompts 600-604 contain an anchor gene or anchor compound, text rating instructions, and/or context generation instructions. The compound exploration initiation system 102 utilizes the language machine learning model 606 to generate rating metrics 608-612 from the digital text prompts 600-604.

As discussed above, the rating metrics 608-612 can include a variety of different values or metrics corresponding to different text rating instructions. Thus, for example, the language machine learning model 606 generates the rating metric 608 according to a first text rating instruction (e.g., a scale from 1 to 10), resulting in a rating metric of 3.2. Similarly, the language machine learning model 606 generate the rating metric 610 according to a second text rating instruction (e.g., a score of 1, 2, 3, or 4) resulting in a rating metric of 4. In addition, the language machine learning model 606 generates the rating metric 612 according to a third rating instruction (e.g., binary yes/no), resulting in a rating metric of “no.”

Further, in some embodiments the compound exploration initiation system 102 combines the rating metrics 608-612 to generate a program rating 614. For instance, the compound exploration initiation system 102 can assign a binary rating metric a score (e.g., “yes”=5 and “no” =0) and further add the binary rating metric with the other rating metrics. In some such instances, the program rating 614 includes adding the rating metrics 608-612. In some instances, the compound exploration initiation system 102 averages the rating metrics 608-612.

As mentioned, in some embodiments, the compound exploration initiation system 102 utilizes various rating metric thresholds to generate the program rating 614. For instance, the compound exploration initiation system 102 can apply a rating metric threshold to each rating metric (e.g., to determine whether each rating metric will receive a passing or failing score). The compound exploration initiation system 102 can also apply a rating metric threshold to a number of passing rating metrics. For instance, the compound exploration initiation system 102 can require a certain number of passing rating metrics (e.g., 4). If the number of passing rating metrics fails to satisfy the threshold (e.g., 4), then the compound exploration initiation system 102 can determine a corresponding program rating (e.g., a failing program rating). If the number of passing rating metrics satisfies the threshold (e.g., 4), then the compound exploration initiation system 102 can determine a corresponding program rating (e.g., a passing program rating). In one or more embodiments, for each rating metric that satisfies an initial rating metric threshold, the compound exploration initiation system 102 can add one point to the program rating 614.

In some instances, the compound exploration initiation system 102 can establish that at least a majority of the rating metrics has to satisfy the rating metric threshold to generate a favorable program rating. While in some instances, the compound exploration initiation system 102 can establish that only one rating metric has to satisfy the predetermined threshold to generate a favorable program rating.

In some implementations, rating metric threshold includes a combined threshold (e.g., after combining one or more rating metrics). For instance, the disclosed system can establish the rating metric threshold as 4 for the combination of three rating metrics. If the average score after combining three metrics fails to exceed 4, then the program rating generated by the disclosed system indicates to not move forward with a compound exploration program.

In one or more embodiments, the compound exploration initiation system 102 utilizes weights for the rating metrics 608-612. For example, the compound exploration initiation system 102 assigns a 60% weight to the rating metric 608, a 20% weight to the rating metric 610, and a 20% weight to the rating metric 612. Further, in one or more embodiments, the compound exploration initiation system 102 utilizes the weight assigned to the rating metrics 608-612, to generate the program rating 614. For instance, if the program rating 614 is scaled from 0-5, if the rating metric 608 (also scaled from 0-5) is a 5, the lowest score the program rating 614 could be is a 3 (e.g., 60% of the total).

In some implementations, compound exploration initiation system 102 learns weights to apply to various rating metrics in generating a program rating. For instance, the compound exploration initiation system 102 can identify what anchor genes or anchor compounds are identified in subsequent compound exploration programs. The compound exploration initiation system 102 can adjust the weights to emphasize those rating metrics corresponding to these anchor genes or anchor compounds.

In some implementations, the compound exploration initiation system 102 automatically initiates a compound exploration program based on the program rating 614. For example, the compound exploration initiation system 102 can initialize additional machine learning analysis (e.g., additional phenomic digital images) for an anchor gene or anchor compound based on the program rating 614.

In one or more embodiments, the compound exploration initiation system 102 provides the program rating 614 to an administrator device to determine whether to initiate one or more compound exploration programs. In some embodiments a “compound exploration program” includes a process of identifying and selecting potential chemical compounds or molecules for development into new or enhanced drugs or agents. For instance, a compound exploration program includes utilizing the anchor gene involved in an underlying disease and testing many compounds to identify how the compounds interact with the specific anchor. Additionally, compound exploration programs involve optimizing identified compounds and analyzing results of the compounds applied to the specific anchor. Furthermore, as used herein, the term compound can include small molecules or large molecules. Thus, a compound exploration program includes small molecule (e.g., molecules below a threshold size, such as smaller than antibodies) and large molecules (e.g., molecules above a threshold size, such as larger than antibodies). A compound exploration program can relate to a variety of therapeutics and biological relationships, including antibodies, antibody drug conjugates, proteolysis-targeting chimeras (e.g., PROTACS), other targeting chimeras, soluble factors, and RNA therapeutics.

For instance, in some embodiments the compound exploration initiation system 102 utilizes the program rating 614 to determine to initiate an industrial program generation (IPG) process. To illustrate, IPG includes (i) a hit selection to identify statistically strong connections in a biological map to patient-informed phenotypes, (ii) phenomic confirmation (e.g., promising actives are confirmed by automated similarity and concentration-response analytics), (iii) Trekseq confirmation (e.g., compound and gene relationships are confirmed with transcriptomics in the map background), and (iv) Structure-Activity Relationship (SAR) confidence (e.g., actives that behave as a series are identified, and an automated recommendation for expansion is identified).

Moreover, in some embodiments the compound exploration initiation system 102 utilizes the program rating 614 to determine to initiate an industrialized compound generation (ICG) process. For instance, ICG applies to steps subsequent to IPG. Further, in some embodiments ICG includes rapidly searching and expanding from potential hit series in the chemical space (e.g., identified at the IPG stage) and testing the potential hits with various analytical tests (e.g., SAR screens).

As discussed above, in some embodiments the compound exploration initiation system 102 generates a program rating from a language machine learning model and additional data sources. FIG. 7 illustrates the compound exploration initiation system 102 generating multiple rating metrics from various sources (e.g., language machine learning models and databases) to further generate a program rating in accordance with one or more embodiments.

As previously discussed in FIG. 6, the compound exploration initiation system 102 utilizes a language machine learning model 700 to generate a rating metric. As shown in FIG. 7, the compound exploration initiation system 102 utilizes the language machine learning model 700 to generate a previous analysis rating metric 712 and a gene impact rating metric 714.

As used herein, a previous analysis rating metric, refers to a value, score, measure or indication of historical investigation, inquiry, or examination. In particular, a previous analysis rating metric can include a value within a range indicating the extent to which a biological relationship has previously been examined or researched (e.g., the extent to which a compound has been analyzed for its impact on cancer). For example, the previous analysis rating metric can include a measure of previous model validation (e.g., preclinical validation utilizing one or more previous models), therapy availability (e.g., oncology therapy availability or an indication of cancer unmet need), compound availability (e.g., oncology compound availability or an indication of the competitive landscape of compounds for treating cancer or some other disease), or relationship analysis (e.g., known relationship between genes and/or compounds and/or whether the biological relationship is novel). The compound exploration initiation system 102 can generate a previous analysis digital text prompt that includes previous analysis rating instructions (e.g., instructions for generating a previous analysis rating metric). To illustrate, the compound exploration initiation system 102 can generate a digital text prompt that includes previous analysis rating instructions identifying a particular rating scale (e.g., from 0-10). A previous analysis digital text prompt can also include previous analysis contextual instructions (e.g., return or summarize previous research or articles regarding the predicted biological relationship).

Similarly, as used herein, a gene impact rating metric refers to a value, score, measure, or indication of activity, expression, relevance, effect, or influence of a gene. In particular, a gene impact rating can include a measure of gene expression (e.g., oncology expression), gene impact direction (e.g., oncology direction), or toxicity. The compound exploration initiation system 102 can generate a gene impact digital text prompt that includes gene impact rating instructions (e.g., instructions for generating a gene impact rating metric). To illustrate, the compound exploration initiation system 102 can generate a gene impact digital text prompt that includes gene impact rating instructions identifying a particular rating scale (e.g., from 0-5 rate human relevance of this gene with regard to a particular disease). A gene impact digital text prompt can also include gene impact contextual instructions (e.g., return or summarize the manner in which this gene impacts a particular disease).

Thus, as illustrated, the compound exploration initiation system 102 utilizes the language machine learning model 700 to generate the previous analysis rating metric 712 and the gene impact rating metric 714. For instance, the the previous analysis rating metric 712 can include a rating metric for the level of previous research for the anchor gene or anchor compound 502. Similarly, the gene impact rating metric 714 can include determining a disease connection source for a anchor gene (e.g., an association with cancer).

As illustrated, the compound exploration initiation system 102 utilizes the language machine learning model 700 to generate other rating metrics. For example, the language machine learning model 700 generates a rating metric 722 (and the rating metrics 724-726). For instance, the rating metric 722 can include a tractability/druggability rating metric.

As used herein, the term tractability rating metric (or druggability rating metric), refers to a value, score, measure, or indication of influence of compounds or drugs. For example, a tractability rating metric includes a measure of influence of compounds or drugs with regard to a particular disease or biological activity. Thus, a tractability rating metric includes a measure of impact of a drug or compound in treating a disease (e.g., feasibility of treating a disease using a compound). The compound exploration initiation system 102 can generate a tractability digital text prompt that includes tractability rating instructions (e.g., instructions for generating a tractability rating metric). To illustrate, the compound exploration initiation system 102 can generate a tractability digital text prompt that includes tractability rating instructions identifying a particular rating scale (e.g., from 0-5 rate tractability or druggability of a particular disease or biological activity). A tractability digital text prompt can also include tractability contextual instructions (e.g., describe one or more sources for the tractability rating metric).

Further, FIG. 7 shows the compound exploration initiation system 102 also performing a database query 702. For example, compound exploration initiation system 102 queries a cancer dependency map. The compound exploration initiation system 102 can generate or access a cancer dependency map that identifies or describes genetic dependencies in cancer and aims to provide information about essential genes and genetic vulnerabilities in cancer cell lines. As shown, from the database query 702, the compound exploration initiation system 102 generates a rating metric 716 by parsing the cancer dependency map to identify for a anchor gene a likelihood of oncological properties. Specifically, the cancer dependency map can illustrate a relationship about how a genetic can affect the growth and survival of cancer cells. Accordingly, in some embodiments the compound exploration initiation system 102 generates the rating metric 716 (e.g., a gene impact score indicating cancer dependency) by extracting a likelihood or other cancer impact indicator related to the anchor gene.

Moreover, FIG. 7 shows the compound exploration initiation system 102 performing a clinical genomics query 704. In particular, the compound exploration initiation system 102 queries a clinical genomics database comprising clinical genomics data. For example, clinical genomics data includes genetic and genomic information relevant to clinical applications to interpret individual genetic information (e.g., derived from DNA sequencing). As shown, from the clinical genomics query 704, the compound exploration initiation system 102 generates a rating metric 718 by parsing the clinical genomics data with a anchor gene to identify a genetic basis for a disease, to identify potential drug anchors, and to facilitate the further development of drugs that anchor specific genes. Accordingly, from a clinical genomics database, the compound exploration initiation system 102 generates the rating metric by extracting an indication for a anchor gene (or anchor compound) for a specific query related to a disease, gene, or a compound.

Further, FIG. 7 shows the compound exploration initiation system 102 performing a biological and genomic database query 706. For example, the compound exploration initiation system 102 queries a genomic database that includes various biological and genetic data related to potential drug anchors and diseases. For instance, the genomic database can include genetic studies, genomic data, and disease associations. As shown, from the biological and genomic database query 706, the compound exploration initiation system 102 generates a rating metric 720 by parsing through a genomic database to extract an indicator of an anchor gene being related to a specific biological substrate.

Moreover, for the tractability determination, the compound exploration initiation system 102 further performs a first database query 708 from the cancer dependency map, which was discussed above, and a second database query 710 from a cancer database. For example, the cancer database can include data related to drug discovery, data visualization for cancer related biological substrates, ongoing cancer research, and other data related to medical compounds to anchor potential cancer substrates. As such, the cancer dependency map and the cancer database can contain some overlaps in data, which helps the compound exploration initiation system 102 reinforce findings related to oncological properties of an anchor gene. As shown from the first database query 708 and 710, the compound exploration initiation system 102 generates the rating metric 724 in a same or similar manner as discussed above and the rating metric 726 by parsing the cancer database to extract a correlation or indicator from the cancer related biological substrates, ongoing cancer research, and other data related to medical compounds for an anchor gene.

Similar to the principles discussed above in FIG. 6, the compound exploration initiation system 102 generates a combined score 728 from the rating metrics 712-720. To illustrate, the compound exploration initiation system 102 can combine any subset of the rating metrics shown in FIG. 7 to determine the combined score 728. As described in relation to FIG. 6, the compound exploration initiation system 102 can combine rating metrics utilizing various approaches, including summing, averaging, learned weighted averaging, or by applying various thresholds. For instance, the compound exploration initiation system 102 can combine the previous analysis rating metric 712 and the gene impact rating metric 714, and if the combination satisfies a predetermined threshold, then the compound exploration initiation system 102 indicates a higher combined score (e.g., on a scale from 0-5, a more favorable score could be greater than 3). Further, the compound exploration initiation system 102 can combine additional subsets (rating metric 716 with rating metric 714, rating metric 716 with 718, rating metric 718 with rating metric 714, etc.) to determine the combined score 728.

Likewise, by combining the rating metrics 722-726, the compound exploration initiation system 102 generates a combined score 730 to determine whether a predetermined threshold is satisfied. To illustrate, the compound exploration initiation system 102 takes the combined score 728 and the combined score 730 and further determines a program rating 732. For instance, the program rating 732 can indicate whether a predetermined threshold was satisfied from a combination of rating metrics (or for individual rating metrics). For instance, the compound exploration initiation system 102 can indicate the program rating 732 as favorable if one of the rating metrics satisfies the predetermined threshold.

Although the above discussion describes utilizing a predetermined threshold to generate the program rating 732 (e.g., based on the combined scores), in some embodiments the compound exploration initiation system 102 utilizes the combined score(s) as the program rating 732 without the predetermined threshold. For instance, the compound exploration initiation system 102 provides the program rating 732 to an administrator computing device and a user of the administrator computing device can determine whether to initiate one or more compound exploration programs.

Furthermore, although FIG. 7 shows a specific number of database queries and rating metrics, in one or more embodiments the compound exploration initiation system 102 utilizes a different number of sources to generate rating metrics, combined scores, and a program rating. For instance, the compound exploration initiation system 102 can reduce the number of database queries and focus on rating metrics from the language machine learning model. In additional instances, the compound exploration initiation system 102 can focus on rating metrics from other databases (e.g., not including the language machine learning model).

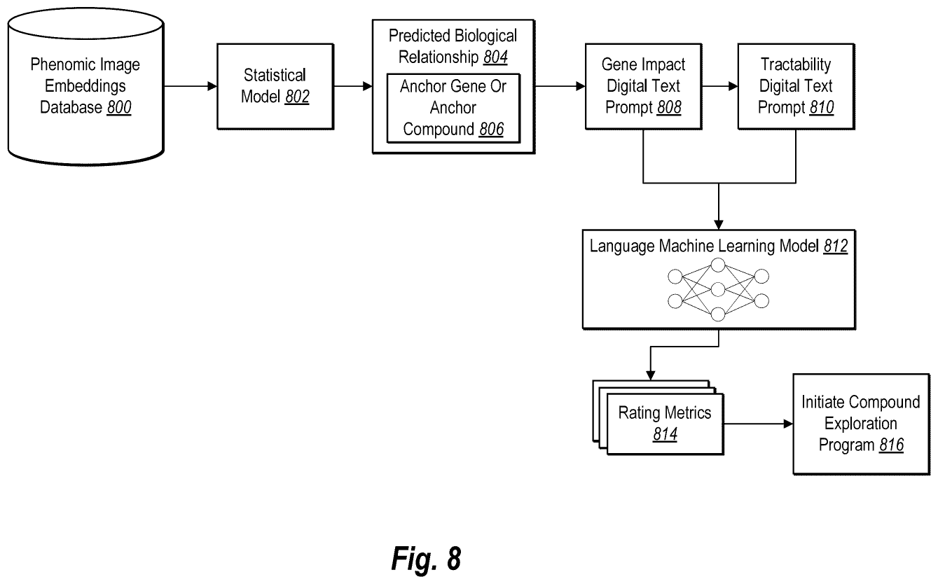

As mentioned above, in some embodiments the compound exploration initiation system 102 utilizes a particular digital text prompts to determine whether to initiate compound exploration programs. FIG. 8 illustrates utilizing an example set of digital text prompts with a language machine learning model in accordance with one or more embodiments.

For example, FIG. 8 illustrates identifying a predicted biological relationship 804 from a phenomic image embeddings database 800 by utilizing a statistical model 802 (e.g., as described in relation to FIGS. 2-4). Furthermore, FIG. 8 shows the predicted biological relationship 804 includes a anchor gene or anchor compound 806. Moreover, from the predicted biological relationship 804, the compound exploration initiation system 102 generates a gene impact digital text prompt 808 and a tractability digital text prompt 810 (e.g., as described in relation to FIG. 5).

In one or more embodiments, the gene impact digital text prompt 808 includes a prompt with text rating instructions for the language machine learning model 812 to rate a gene impact for a particular gene (e.g., the anchor gene). For instance, as mentioned previously a gene impact can include an effect, significance, or importance of a specific gene or variant of the gene in relation to various aspects. To illustrate, the gene impact can include the significance or importance to human relevance, gene function or activity, and gene toxicity signals.

For example, in some embodiments, the gene impact digital text prompt 808 can include a gene expression digital text prompt (e.g., a prompt related to expression of a gene with regard to a biological activity/disease), a gene impact direction digital text prompt (e.g., a prompt related to the functional significance of a gene such as its particular directional role in different cellular processes or biochemical pathways such as whether the gene is considered an oncogene or a suppressor gene), or a gene toxicity digital text prompt (e.g., a prompt related to a gene's role in toxicity such as how a gene's activity or expression relates to toxic effects).

For instance, an oncology expression digital text prompt (e.g., human relevance) can include a prompt as follows: “I will give you a gene and a list of cancer indications in humans. Supply a score and information about the gene's relevance to each of the indications. {gene} scoring rubric: 0—weak relevance of {gene} in cancer indication; 1—some evidence of altered expression of {gene} in cancer; 2—target mutation in the cancer indication; 3—putative target.” Moreover, in some embodiments, the oncology expression digital text prompt can further include a context generation prompt. To illustrate, the context generation prompt can include (in addition to the rating metric e.g., score), “with the score please provide evidence linking {gene} to cancer development and also evidence that modulation of {gene} slows growth.”

Further, an oncology direction digital text prompt (e.g., direction) can include a prompt as follows: “I will provide a gene, supply information about this gene in the following format. Give a confidence score from 0-5 classifying {gene} in each of the following categories: oncogene, tumor suppressor, tumor dependency, driver of drug resistance, loss in drug resistance. Please format the response as follows: {gene}; score for each of the categories.”

Moreover, a gene toxicity digital text prompt (e.g., toxicity) can include a prompt as follows: “Please provide information about how well validated a {gene} is linked to toxicity. For each {gene}, provide information on the quality and reproducibility of evidence linking the gene to toxicity. Assign a score from 0.0-5.0 based on the amount of evidence that validates the gene linked to toxicity.”

In one or more embodiments, the tractability digital text prompt 810 includes a prompt with text rating instructions for the language machine learning model to rate compound tractability. For instance, the tractability digital text prompt includes text for generating a tractability rating metric indicating druggability of a particular biological activity or disease. Specifically, as discussed, druggability includes a likelihood that a specific target can be modulated by a drug.

To illustrate, in one or more embodiments, the tractability digital text prompt 810 includes a compound tractability digital text prompt. To illustrate, the tractability digital text prompt 810 can include a prompt as follows: “I will give you a gene, supply a score and information about the difficulty of developing a drug targeting this gene. Please format the response as follows: {gene}; score. Information regarding the difficulty.”

As shown in FIG. 8, the compound exploration initiation system 102 provides the gene impact digital text prompt 808 and the tractability digital text prompt 810 to the language machine learning model 812 which further generates rating metrics 814. As shown, from the rating metrics 814, the compound exploration initiation system 102 further determines to initiate compound exploration program 816.

In one or more embodiments, in response to the compound exploration initiation system 102 initiating one or more compound exploration programs, the compound exploration initiation system 102 generates an additional processed biological representation. For instance, the compound exploration initiation system 102 generates an additional processed biological representation to begin downstream analysis tasks for the compound exploration program. The additional processed biological representations can include, for example, additional phenomic image embeddings generated from additional phenomic digital images. The additional processed biological representations can also include additional machine learning binding predictions between compounds and proteins. To illustrate, in some embodiments the compound exploration initiation system 102 generates the additional biological machine learning model for use in IPG and/or ICG processes discussed above.

Although the above description in relation to FIG. 8 provides specific example prompts regarding gene impact and tractability, in one or more embodiments the compound exploration initiation system 102 utilizes different types of prompts and rating metrics. For instance, the compound exploration initiation system 102 can utilize various previous analysis digital text prompts (and corresponding previous analysis rating metrics) and/or tractability digital text prompts (and corresponding tractability rating metrics). Additional detail is now provided regarding these text prompts and corresponding examples.

For instance, although not shown in FIG. 8, in some embodiments the compound exploration initiation system 102 utilizes a previous analysis digital text prompt. For example, the previous analysis digital text prompt includes a prompt for exploring historical experimental investigation or analysis of a anchor gene or anchor compound. For instance, the previous analysis digital text prompt includes, a previous model validator digital text prompt (e.g., a text prompt for generating previous model validator rating metrics indicating a measure of previous models validating a particular biological relationship, anchor gene, or anchor compound), a therapy availability digital text prompt (e.g., a text prompt for generating therapy availability digital rating metrics indicating the accessibility and availability of therapeutic treatments or interventions such as medications, medical procedures or other related therapies to treat certain diseases or conditions), a compound availability digital text prompt (e.g., a text prompt for generating compound availability rating metrics indicating the availability of specific chemical compounds for use in a specific biological application), or a relationship analysis digital text prompt (e.g., a text prompt for generating relationships analysis rating metrics indicating previous analysis of a relationship between two or more genes or a gene or compound or compounds, etc.).

For example, in some embodiments the previous model validator digital text prompt includes an oncology validation digital text prompt. For instance, the oncology validation digital text prompt includes a text prompt for generating oncology validation rating metrics indicating a measure of previous models validating a relationship between a gene/compound and cancer.

To illustrate, an oncology validation digital text prompt can include a digital text prompt as follows: “I will provide a gene, supply information about this gene in the following format. Here is a scoring rubric from 0-4 for {gene}. 0—no in-vivo or in-vitro data; 1—in-vitro evidence showing target link; 2—single in-vitro and in-vivo study; 3—in-vivo data in more than two models; 4—in-vivo data in multiple models in greater than two peer reviewed studies. Please format the response as follows: {gene}; 0-4 score based on the rubric.”

In some embodiments, a therapy availability digital text prompt can include an oncology therapy availability text prompt (which further includes cancer indication unmet need). For instance, the oncology therapy availability text prompt can include a text prompt for generating an oncology therapy rating metric indicating a measure of existing cancer treatments/therapies.

For example, an oncology therapy availability digital text prompt can include a prompt as follows: “I will provide a human cancer indication and you will supply a score and information about the unmet need in this indication. Here is the cancer indication: {indication}. Scoring rubric: 0—curative treatments exist; 1—low unmet need, treatments available; 2—medium low unmet need, multiple lines of therapy available; 3—medium unmet need, some targeted treatments available; 4—medium high unmet need, treatment options limited; 5—high unmet need, no treatments available.” Moreover, in some instances, the oncology therapy availability digital text prompt can include a context generation prompt. For example, the context generation prompt can include “score+estimated number of new {country} cases per year, estimated number of {country} deaths per year, and an explanation for the reason of the score.”

In addition, a compound availability digital text prompt can include an oncology compound availability text prompt. For instance, the compound availability text prompt can include a text prompt for generating an oncology availability rating metric indicating a measure of cancer treating compounds (e.g., already existing or utilized in the competitive landscape).

For example, in some instances, the oncology compound availability digital text prompt can include a prompt as follows: “I will give you a human cancer indication and you will supply a score and information about the presence, phase, and progress of efforts targeting the cancer indication. Cancer {indication}. Scoring rubric: 0—high, >2 competitors with approved drugs for the {indication}; 1—medium high, >4 competitors in clinical trials; 2—medium, <4—clinical trials; 3—medium low, <4 competitors in early phase clinical trials; 4—low, 2 competitors in early phase clinical trials; 5—very low, no competitors in clinical trials.” Moreover, the oncology compound availability digital text prompt can include a context generation prompt that includes “score+the number of programs and the latest phase reached in a clinical trial.”

In some embodiments, a relationship analysis digital text prompt includes a known relationship digital text prompt (e.g., a prompt for measuring novel relationships between genes or between a gene and a compound). For instance, the relationship analysis digital text prompt includes a text prompt for generating a relationship analysis rating metric indicating a measure of novelty for a particular relationship between genes, drug candidates, and/or biological target. For instance, a known relationship digital text prompt can include a prompt as follows: “I will give you a gene pair, tell me whether the two genes are known to be biologically related (in the same pathway). {gene 1} and {gene 2}.”

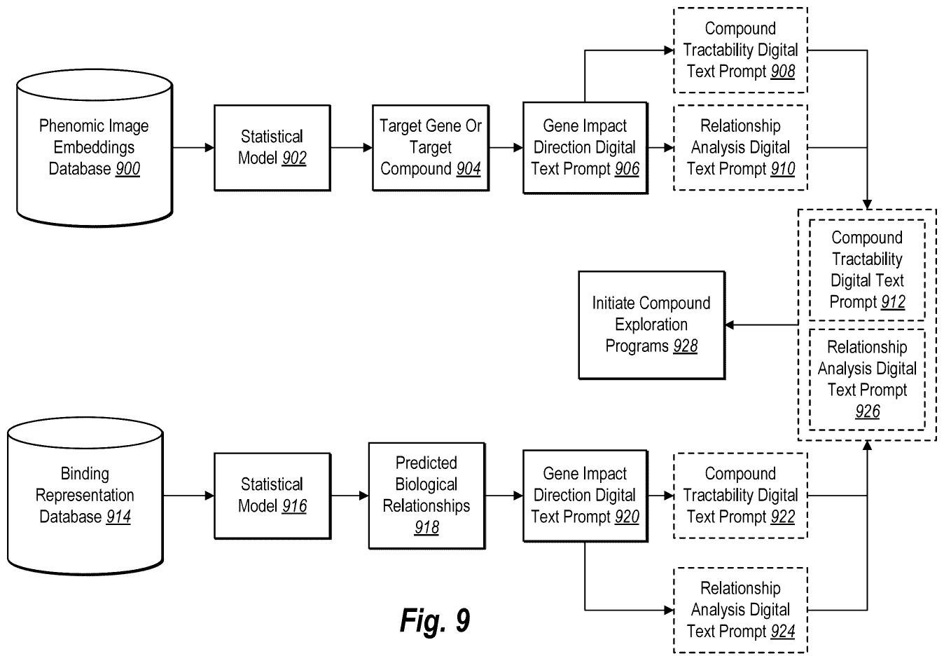

Although FIG. 8 illustrates a particular set of prompts for generating rating metrics, the compound exploration initiation system 102 can utilize a variety of prompts in a variety of different orders or workflows to generate rating metrics and program ratings for initiating compound exploration programs. For example, FIG. 9 illustrates a dual digital text prompt stream/workflow for determining whether to initiate compound exploration programs in accordance with one or more embodiments.

In one or more embodiments, the compound exploration initiation system 102, parses through the phenomic image embeddings database 900 to identify a plurality of predicted biological relationships by comparing phenomic image embeddings with one another. For example, “the top stream” of FIG. 9 shows a phenomic image embeddings database 900 and the compound exploration initiation system 102 utilizing a statistical model 902 to identify a anchor gene or anchor compound 904.

As shown, the compound exploration initiation system 102 can utilize multiple data streams to identify related gene-compound/protein-compound interactions and further generate different digital text prompts for the language machine learning model to generate rating metrics.

Specifically, the compound exploration initiation system 102 can identify a related protein to the anchor gene and utilize protein binding predictions to analyze a predicted biological relationship (and generate rating metrics utilizing a language machine learning model). For instance, the compound exploration initiation system 102 can identify a protein synthesized by an anchor gene. In some such instances, the compound exploration initiation system 102 can further utilize a binding representation database 914 to analyze protein-compound interactions for this corresponding protein to determine a predicted biological relationship. Moreover, the compound exploration initiation system 102 can analyze this predicted biological relationship utilizing a prompts and a language machine learning model to generate rating metrics. In other words, the compound exploration initiation system 102 identifies protein-compound interactions in the binding representation database 914 and utilizes these interactions as a predicted biological relationship for generating rating metrics (in parallel with the predicted biological relationships generated from the phenomic image embeddings database 900). In other words, FIG. 9 illustrates using different data streams to generate different types of rating metrics (from various digital text prompts) to determine how to proceed for a compound exploration program.

As shown in the top stream, the compound exploration initiation system 102 generates a gene impact direction digital text prompt 906, then the compound exploration initiation system 102 can generate a compound tractability digital text prompt 908 (e.g., a druggability digital text prompt) and/or a relationship analysis digital text prompt 910. Accordingly, the compound exploration initiation system 102 can utilize rating metrics generated from each of the digital text prompts to adjust/modify digital text prompts used in a parallel data stream (e.g., the bottom data stream).

As shown in “the bottom stream” of FIG. 9, the compound exploration initiation system 102 utilizes the binding representation database 914 and a statistical model 916 to identify a predicted biological relationship 918 (e.g., based on the anchor gene synthesizing a specific protein, the compound exploration initiation system 102 identifies a protein-compound interaction). From the predicted biological relationship 918, the compound exploration initiation system 102 generates a gene impact direction digital text prompt 920. Subsequent to the gene impact direction digital text prompt 920, the compound exploration initiation system 102 generates a compound tractability digital text prompt 922 and/or a relationship analysis digital text prompt 924.

In utilizing the digital text prompts shown in FIG. 9, the compound exploration initiation system 102 can identify compounding data relationships from multiple initiating sources (e.g., from both the phenomic image embeddings database 900 and from the binding representation database 914). For instance, utilizing multiple data streams the compound exploration initiation system 102 determines multiple rating metrics to streamline the process of validating compound exploration hypotheses.

As shown, following the compound tractability digital text prompt 908 and/or the relationship analysis digital text prompt 910, the compound exploration initiation system 102 further generates a compound tractability digital text prompt 912 and/or a relationship analysis digital text prompt 926 to generate additional rating metrics related to the anchor gene or anchor compound 904 and the predicted biological relationships 918.

As shown, from the series of digital text prompts submitted to a language machine learning model, the compound exploration initiation system 102 generates various rating metrics and a program rating to determine to perform the act 928 of initiating compound exploration programs.

Although not illustrated in FIG. 9, in one or more embodiments, the compound exploration initiation system 102 utilizes a combination algorithm at each step of the sequence of digital text prompts shown in FIG. 9. For instance, the combination algorithm can include a decision tree for determining dependencies and threshold rating metrics linked to generating specific digital text prompts. For example, the combination algorithm as a decision tree includes weights assigned to digital text prompts for lower scoring rating metrics (e.g., below 3) and for high scoring rating metrics (e.g., above 3). To illustrate, the decision tree can include if the gene impact direction digital text prompt 906 has a rating metric greater than 3 then initiate a relationship analysis digital text prompt 910. Moreover, in some instances the decision tree can include if the gene impact direction digital text prompt 906 has a rating metric of 5 then initiate an oncology therapy availability digital text prompt and an oncology compound availability digital text prompt. Thus, the compound exploration initiation system 102 can utilize some rating metrics as filters to determine whether to generate and utilize additional digital text prompts with a language machine learning model.

Further, in some instances the compound exploration initiation system 102 can utilize the combination algorithm to receive as input multiple rating metrics corresponding to multiple digital text prompts and determine a subsequent digital text prompt. Specifically, in some instances the compound exploration initiation system 102 assigns weights to different digital text prompts, and for a combination of rating metrics below a certain number, the compound exploration initiation system 102 identifies a subsequent digital text prompt with a lower weight. Conversely, for a combination of rating metrics above a certain number, the compound exploration initiation system 102 identifies a subsequent digital text prompt with a higher weight. Moreover, in some instances the compound exploration initiation system 102 utilizes the combination algorithm to generate the program rating from combining multiple rating metrics.

Although not shown in FIG. 9, in one or more embodiments, prior to performing the act 928 to initiate compound exploration programs, the compound exploration initiation system 102 generates or performs additional steps. For instance, the compound exploration initiation system 102 generates a notification to the administrator computing devices to order specific profiles or compounds related to the anchor gene or anchor compound 904 (based on the predicted biological relationships 918 from the binding representation database 914). Further, in some embodiments the compound exploration initiation system 102 determines to drive Structure-Activity Relationship (SAR) confidence (e.g., actives that behave as a series are identified, and an automated recommendation for expansion is identified) prior to initiating compound exploration programs.

Furthermore, although FIG. 9 shows a specific ordering of digital text prompts, in one or more embodiments, the compound exploration initiation system 102 omits, adds, or reorders the digital text prompts. Moreover, rather than just a dual stream, in some embodiments the compound exploration initiation system 102 could integrate additional databases such as multiomic databases (e.g., proteomics, metabolomics, and transcriptomics). For instance, the compound exploration initiation system 102 could simultaneously be identifying various biological relationships from different multiomic databases, the phenomic image embeddings database 900, and the binding representation database 914 to generate various digital text prompts and initiate compound exploration programs. Moreover, in some embodiments the compound exploration initiation system 102 utilizes the outputs (e.g., the rating metrics) related to the phenomic image embeddings database 900 as inputs to identify the predicted biological relationships 918 from the binding representation database 914 (e.g., or other multiomics databases).

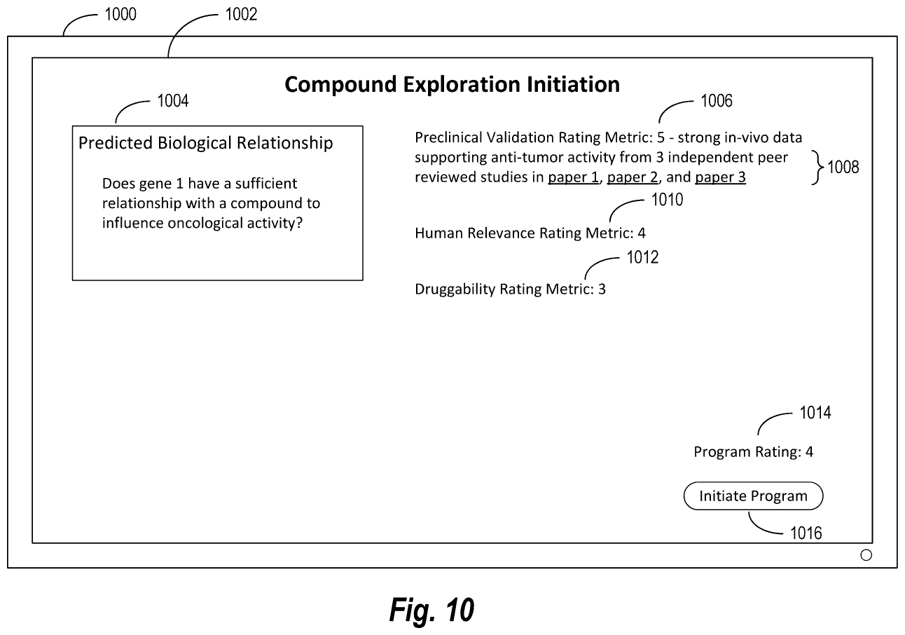

In some implementations, the compound exploration initiation system 102 generates user interfaces for efficiently displaying rating metrics, contextual information, and/or program ratings in initiating a compound exploration program. FIG. 10 illustrates an example graphical user interface shown on an administrator computing device 1000. In one or more embodiments, the administrator computing device 1000 includes a computing device associated with an administrator overseeing compound exploration programs. In particular, in some embodiments the administrator computing device 1000 indicates to the compound exploration initiation system 102 an anchor gene or anchor compound and to generate a digital text prompt including the anchor gene or anchor compound.

As shown, FIG. 10 illustrates the administrator computing device 1000 and a graphical user interface 1002. As shown in FIG. 10, the compound exploration initiation system 102 causes the graphical user interface 1002 to display a predicted biological relationship 1004 that reads “does gene 1 have a sufficient relationship with a compound to influence oncological activity?” In some implementations, the compound exploration initiation system 102 automatically generates the predicted biological relationship 1004. In some implementations, the compound exploration initiation system 102 identifies the predicted biological relationship 1004 based on user input (e.g., a user interaction with a text field or other user input element for selecting the predicted biological relationship). In some embodiments, the compound exploration initiation system 102 generates a relationship table or graph of similarity measures between genes and/or compounds and determines the predicted biological relationship based on user interaction with the table or graph (e.g., selection of a particular relationship or similarity measure).

Furthermore, the compound exploration initiation system 102 causes the graphical user interface 1002 to display a first rating metric 1006, a second rating metric 1010, and a third rating metric 1012. For instance, the compound exploration initiation system 102 receives the predicted biological relationship and identifies a set of digital text prompts to send to the language machine learning model. Further, in some such instances the compound exploration initiation system 102 generates rating metrics for the set of digital text prompts utilizing the language machine learning model. Moreover, in such instances the compound exploration initiation system 102 causes the graphical user interface 1002 to provide for display the rating metrics obtained from the language machine learning model.

As shown, the first rating metric 1006 reads” preclinical validation rating metric: 5-strong in vivo data supporting anti-tumor activity from 3 independent peer reviewed studies in paper 1, paper 2, and paper 3.” As shown, the text following the first rating metric 1006 includes context 1008 returned with the rating metric in response to context generation instructions. Further, the underlined paper 1, paper 2, and paper 3 can indicate links to the cited papers. For instance, the compound exploration initiation system 102 receives the predicted biological relationship and generates a set of digital text prompts that includes context generation instructions. Moreover, in the context generation instructions instruct the language machine learning model to return published papers related to the rating metrics. Accordingly, the compound exploration initiation system 102 receives from the language machine learning model the rating metrics and corresponding published papers that support the rating metrics. Thus, the compound exploration initiation system 102 causes the graphical user interface 1002 to display the context 1008 obtained from the language machine learning model.

Moreover, as shown, the second rating metric 1010 reads “human relevance rating metric: 4” and the third rating metric 1012 reads “druggability rating metric: 3.” For instance, the compound exploration initiation system 102 identifies the predicted biological relationship for a anchor gene and identifies a corresponding human relevance digital text prompt. Moreover, in some such instances the compound exploration initiation system 102 sends the human relevance digital text prompt to the language machine learning model and receives a rating metric of 4 (e.g., which indicates in the example given above a putative target with known significance to cancer). Likewise, the compound exploration initiation system 102 receives the druggability rating metric, in a similar manner as just described. Specifically, the compound exploration initiation system 102 receives these rating metrics and causes the graphical user interface 1002 to display the rating metrics obtained from the language machine learning model.

As further shown, the compound exploration initiation system 102 further causes the graphical user interface 1002 to display a program rating 1014 which reads “program rating: 4.” For instance, the compound exploration initiation system 102 receives the rating metrics 1006, 1010, and 1012 from the language machine learning model and further combines the rating metrics to determine a program rating. As described above, in some embodiments, the compound exploration initiation system 102 utilizing a program rating model (e.g., combination model) to combine individual rating metrics and determine the program rating 1014. Further, after generating the program rating 1014, the compound exploration initiation system 102 causes the graphical user interface 1002 to display the program rating 1014.

In addition to showing the program rating, the compound exploration initiation system 102 also causes the graphical user interface 1002 to provide an element 1016 that reads “initiate program.” In some embodiments, selecting the element 1016 causes the compound exploration initiation system 102 to trigger one or more compound exploration program(s) related to the predicted biological relationship 1004 (e.g., such as generating an additional machine learning representation for downstream analysis).

As mentioned above, in some embodiments the compound exploration initiation system 102 scales the validation of hundreds of thousands to millions of predicted biological relationships by automatically querying the language machine learning model with digital text prompts related to the predicted biological relationships. Although not shown in FIG. 10, in one or more embodiments, a user corresponding with the administrator computing device 1000 can interact with the language machine learning model. Specifically, the compound exploration initiation system 102 provides to the administrator computing device a chat environment integrated with the language machine learning model. In some embodiments the compound exploration initiation system 102 provides a command line prompting environment to query the language machine learning model with an application programming interface call (e.g., API call).

In some implementations, the compound exploration initiation system 102 generates digital text prompts and provides the digital text prompts for display via the administrator computing device 1000. The administrator computing device 1000 can then view and/or modify the text prompts before applying the language machine learning model.

Although not illustrated, in some implementations, the compound exploration initiation system 102 monitors performance of compounds/genes in future programs to improve program initiation. For example, the compound exploration initiation system 102 monitors IPG and/or ICG processes to identify successful compounds that modulate biology. The compound exploration initiation system 102 can then utilize these successful compounds to modify weights, combination algorithms, or parameters to improve program initiation predictions in the future.

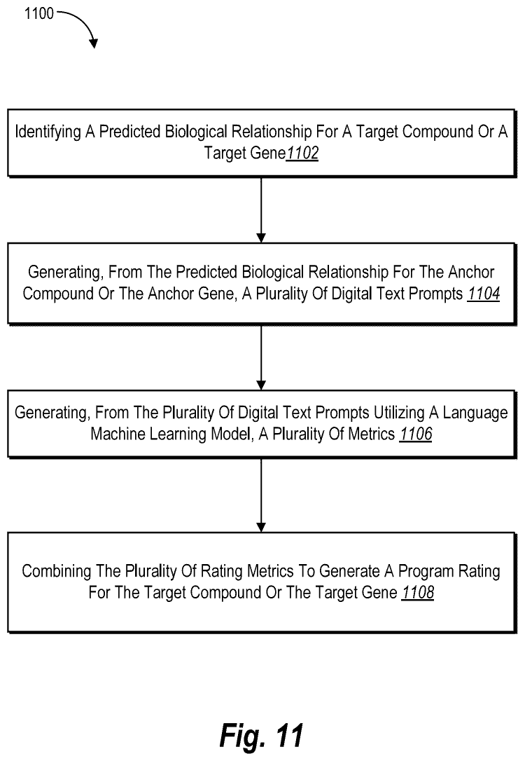

FIGS. 1-10, the corresponding text, and the examples provide a number of different systems, methods, and non-transitory computer readable media for transmitting a compound discovery status update notification to one or more computing devices. In addition to the foregoing, embodiments can also be described in terms of flowcharts comprising acts for accomplishing a particular result. For example, FIG. 11 illustrates a flowchart of an example sequence of acts in accordance with one or more embodiments.

While FIG. 11 illustrates acts according to some embodiments, alternative embodiments may omit, add to, reorder, and/or modify any of the acts shown in FIG. 11. The acts of FIG. 11 can be performed as part of a method (e.g., a computer-implemented method). Alternatively, a non-transitory computer readable medium can comprise instructions, that when executed by one or more processors (e.g., at least one processor), cause a computing device to perform the acts of FIG. 11. In still further embodiments, a system can perform the acts of FIG. 11. Additionally, the acts described herein may be repeated or performed in parallel with one another or in parallel with different instances of the same or other similar acts.

FIG. 11 illustrates an example series of acts 1100 for combining a plurality of rating metrics to generate a program rating. The series of acts 1100 can include acts 1102-1108 of identifying a predicted biological relationship for an anchor compound or an anchor gene; generating, from the predicted biological relationship for the anchor compound or the anchor gene, a plurality of digital text prompts; generating, from the plurality of digital text prompts utilizing a language machine learning model, a plurality of metrics; and combining the plurality of rating metrics to generate a program rating for the anchor compound or the anchor gene.

For example, in one or more embodiments, the acts 1102-1108 include identifying, from a processed biological representation (or biological machine learning representation), a predicted biological relationship for an anchor compound or an anchor gene; generating, from the predicted biological relationship for the anchor compound or the anchor gene, a plurality of digital text prompts, wherein the plurality of digital text prompts comprise the anchor compound or the anchor gene and a plurality of text rating instructions for a language machine learning model; generating, from the plurality of digital text prompts utilizing the language machine learning model, a plurality of rating metrics according to the plurality of text rating instructions; and combining the plurality of rating metrics to generate a program rating for the anchor compound or the anchor gene for initiating one or more compound exploration programs.

In one or more implementations, the series of acts 1100 include generating, utilizing a machine-learning model, a plurality of phenomic image embeddings from a plurality of perturbation images portraying a plurality of cell perturbations; comparing the plurality of phenomic image embeddings to determine a measure of similarity; and identifying the predicted biological relationship from the measure of similarity.

In addition, in one or more implementations, the series of acts 1100 includes identifying compound features corresponding to a compound and protein features corresponding to a protein; generating, utilizing a compound protein-pocket interaction machine-learning model, a machine learning binding representation between the compound and the protein utilizing the compound features and the protein features; and identifying the predicted biological relationship from the machine learning binding representation.

Further, in some implementations, the series of acts 1100 includes identifying a plurality of digital text prompt templates comprising one or more placeholder query fields; and generating the plurality of digital text prompts by populating the one or more placeholder query fields of the plurality of digital text prompt templates based on the anchor compound or the anchor gene.

In one or more implementations, the series of acts 1100 includes generating the plurality of digital text prompts further comprises generating, for the anchor compound or the anchor gene, a gene impact digital text prompt comprising gene impact text rating instructions; and generating the plurality of rating metrics comprises generating, from the gene impact digital text prompt comprising the gene impact text rating instructions utilizing the language machine learning model, a gene impact rating metric indicating a measure of impact corresponding to the target gene.

In addition, in some implementations, the series of acts 1100 includes generating, for the anchor compound or the anchor gene, at least one of: a previous analysis digital text prompt comprising previous analysis text rating instructions indicating a measure of previous analysis of the predicted biological relationship, or a tractability digital text prompt comprising tractability text rating instructions indicating a measure of tractability of impacting the anchor gene utilizing a compound.

Further, in one or more implementations, the series of acts 1100 includes generating a digital text prompt comprising the anchor compound or the anchor gene, a text rating instruction, and a context generation instruction; generating, from the context generation instruction utilizing the language machine learning model, a contextual text description for the anchor compound or the anchor gene; and providing, for display, via a graphical user interface of an administrator computing device, the program rating and the contextual text description.

In addition, in one or more implementations, the series of acts 1100 includes generating the program rating based on determining that a subset of rating metrics of the plurality of rating metrics satisfies a predetermined rating metric threshold.

Further, in one or more implementations, the series of acts 1100 includes providing for display via a graphical user interface of an administrator computing device, the program rating, for the anchor compound or the anchor gene, and the plurality of rating metrics. Moreover, in one or more implementations, the series of acts 1100 includes initiating the one or more compound exploration programs based on the program rating by generating an additional processed biological representation for the anchor compound or the anchor gene.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 12 illustrates a block diagram of an example computing device 1200 that may be configured to perform one or more of the processes described above. One will appreciate that one or more computing devices, such as the computing device 1200 may represent the computing devices described above. In one or more embodiments, the computing device 1200 may be a mobile device (e.g., a mobile telephone, a smartphone, a PDA, a tablet, a laptop, a camera, a tracker, a watch, a wearable device, etc.). In some embodiments, the computing device 1200 may be a non-mobile device (e.g., a desktop computer or another type of client device). Further, the computing device 1200 may be a server device that includes cloud-based processing and storage capabilities.

As shown in FIG. 12, the computing device 1200 can include one or more processor(s) 1202, memory 1204, a storage device 1206, input/output interfaces 1208 (or “I/O interfaces 1208”), and a communication interface 1210, which may be communicatively coupled by way of a communication infrastructure (e.g., bus 1212). While the computing device 1200 is shown in FIG. 12, the components illustrated in FIG. 12 are not intended to be limiting. Additional or alternative components may be used in other embodiments. Furthermore, in certain embodiments, the computing device 1200 includes fewer components than those shown in FIG. 12. Components of the computing device 1200 shown in FIG. 12 will now be described in additional detail.

In particular embodiments, the processor(s) 1202 includes hardware for executing instructions, such as those making up a computer program. As an example, and not by way of limitation, to execute instructions, the processor(s) 1202 may retrieve (or fetch) the instructions from an internal register, an internal cache, memory 1204, or a storage device 1206 and decode and execute them.

The computing device 1200 includes memory 1204, which is coupled to the processor(s) 1202. The memory 1204 may be used for storing data, metadata, and programs for execution by the processor(s). The memory 1204 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1204 may be internal or distributed memory.

The computing device 1200 includes a storage device 1206 includes storage for storing data or instructions. As an example, and not by way of limitation, the storage device 1206 can include a non-transitory storage medium described above. The storage device 1206 may include a hard disk drive (HDD), flash memory, a Universal Serial Bus (USB) drive or a combination these or other storage devices.

As shown, the computing device 1200 includes one or more I/O interfaces 1208, which are provided to allow a user to provide input to (such as user strokes), receive output from, and otherwise transfer data to and from the computing device 1200. These I/O interfaces 1208 may include a mouse, keypad or a keyboard, a touch screen, camera, optical scanner, network interface, modem, other known I/O devices or a combination of such I/O interfaces 1208. The touch screen may be activated with a stylus or a finger.

The I/O interfaces 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1208 are configured to provide graphical data to a display for presentation to a user. The graphical data may be representative of one or more graphical user interfaces and/or any other graphical content as may serve a particular implementation.

The computing device 1200 can further include a communication interface 1210. The communication interface 1210 can include hardware, software, or both. The communication interface 1210 provides one or more interfaces for communication (such as, for example, packet-based communication) between the computing device and one or more other computing devices or one or more networks. As an example, and not by way of limitation, communication interface 1210 may include a network interface controller (NIC) or network adapter for communicating with an Ethernet or other wire-based network or a wireless NIC (WNIC) or wireless adapter for communicating with a wireless network, such as a WI-FI. The computing device 1200 can further include a bus 1212. The bus 1212 can include hardware, software, or both that connects components of computing device 1200 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1200 can include a client device that includes a requester application or a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 104 may include a variety of servers, sub-systems, programs, modules, logs, and data stores. In particular embodiments, the tech-bio exploration system 104 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 104 may also include suitable components such as network interfaces, security mechanisms, load balancers, failover servers, management-and-network-operations consoles, other suitable components, or any suitable combination thereof. In particular embodiments, the tech-bio exploration system 104 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 104 and one or more client devices. An action logger may be used to receive communications from a web server about a user's actions on or off the tech-bio exploration system 104. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 104. A privacy setting of a user determines how particular information associated with a user can be shared. The authorization server may allow users to opt in to or opt out of having their actions logged by the tech-bio exploration system 104 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
identifying, from a processed biological representation, a predicted biological relationship for an anchor compound or an anchor gene;
generating, from the predicted biological relationship for the anchor compound or the anchor gene, a plurality of digital text prompts, wherein the plurality of digital text prompts comprise the anchor compound or the anchor gene and a plurality of text rating instructions for a language machine learning model;
generating, from the plurality of digital text prompts utilizing the language machine learning model, a plurality of rating metrics according to the plurality of text rating instructions; and
combining the plurality of rating metrics to generate a program rating for the anchor compound or the anchor gene for initiating one or more compound exploration programs.

2. The computer-implemented method of claim 1, further comprising generating the processed biological representation to identify the predicted biological relationship by:
generating, utilizing a machine-learning model, a plurality of phenomic image embeddings from a plurality of perturbation images portraying a plurality of cell perturbations;
comparing the plurality of phenomic image embeddings to determine a measure of similarity; and
identifying the predicted biological relationship from the measure of similarity.

3. The computer-implemented method of claim 1, further comprising generating the processed biological representation to identify the predicted biological relationship by:
identifying compound features corresponding to a compound and protein features corresponding to a protein;
generating, utilizing a compound protein-pocket interaction machine-learning model, a machine learning binding representation between the compound and the protein utilizing the compound features and the protein features; and
identifying the predicted biological relationship from the machine learning binding representation.

4. The computer-implemented method of claim 1, wherein generating the plurality of digital text prompts further comprises:
identifying a plurality of digital text prompt templates comprising one or more placeholder query fields; and
generating the plurality of digital text prompts by populating the one or more placeholder query fields of the plurality of digital text prompt templates based on the anchor compound or the anchor gene.

5. The computer-implemented method of claim 1, wherein:
generating the plurality of digital text prompts further comprises generating, for the anchor compound or the anchor gene, a gene impact digital text prompt comprising gene impact text rating instructions; and
generating the plurality of rating metrics comprises generating, from the gene impact digital text prompt comprising the gene impact text rating instructions utilizing the language machine learning model, a gene impact rating metric indicating a measure of impact corresponding to the anchor gene.

6. The computer-implemented method of claim 1, wherein generating the plurality of digital text prompts further comprises:
generating, for the anchor compound or the anchor gene, at least one of:
a previous analysis digital text prompt comprising previous analysis text rating instructions indicating a measure of previous analysis of the predicted biological relationship, or
a tractability digital text prompt comprising tractability text rating instructions indicating a measure of tractability of impacting the anchor gene utilizing a compound.

7. The computer-implemented method of claim 1, wherein generating the plurality of digital text prompts comprises:
generating a digital text prompt comprising the anchor compound or the anchor gene, a text rating instruction, and a context generation instruction;
generating, from the context generation instruction utilizing the language machine learning model, a contextual text description for the anchor compound or the anchor gene; and
providing, for display, via a graphical user interface of an administrator computing device, the program rating and the contextual text description.

8. The computer-implemented method of claim 1, wherein combining the plurality of rating metrics to generate a program rating comprises generating the program rating based on determining that a subset of rating metrics of the plurality of rating metrics satisfies a predetermined rating metric threshold.

9. The computer-implemented method of claim 1, further comprising providing for display via a graphical user interface of an administrator computing device, the program rating, for the anchor compound or the anchor gene, and the plurality of rating metrics.

10. The computer-implemented method of claim 1, further comprising initiating the one or more compound exploration programs based on the program rating by generating an additional processed biological representation for the anchor compound or the anchor gene.

11. A system comprising:
at least one processor; and
at least one non-transitory computer-readable storage medium storing instructions that, when executed by the at least one processor, cause the system to:
identify, from a processed biological representation, a predicted biological relationship for an anchor compound or an anchor gene;
generate, from the predicted biological relationship for the anchor compound or the anchor gene, a plurality of digital text prompts, wherein the plurality of digital text prompts comprise the anchor compound or the anchor gene and a plurality of text rating instructions for a language machine learning model;
generate, from the plurality of digital text prompts utilizing the language machine learning model, a plurality of rating metrics according to the plurality of text rating instructions; and
combine the plurality of rating metrics to generate a program rating for the anchor compound or the anchor gene for initiating one or more compound exploration programs.

12. The system of claim 11, further comprising instructions that, when executed by the at least one processor, cause the system to generate the processed biological representation to identify the predicted biological relationship by:
generating, utilizing a machine-learning model, a plurality of phenomic image embeddings from a plurality of perturbation images portraying a plurality of cell perturbations;
comparing the plurality of phenomic image embeddings to determine a measure of similarity; and
identifying the predicted biological relationship from the measure of similarity.

13. The system of claim 11, further comprising instructions that, when executed by the at least one processor, cause the system to generate the processed biological representation to identify the predicted biological relationship by:
identifying compound features corresponding to a compound and protein features corresponding to a protein;
generating, utilizing a compound protein-pocket interaction machine-learning model, a machine learning binding representation between the compound and the protein utilizing the compound features and the protein features; and
identifying the predicted biological relationship from the machine learning binding representation.

14. The system of claim 11, further comprising instructions that, when executed by the at least one processor, cause the system to generate the plurality of digital text prompts by:
identifying a plurality of digital text prompt templates comprising one or more placeholder query fields; and
generating the plurality of digital text prompts by populating the one or more placeholder query fields of the plurality of digital text prompt templates based on the anchor compound or the anchor gene.

15. The system of claim 11, further comprising instructions that, when executed by the at least one processor, cause the system to generate the program rating based on determining that a subset of rating metrics of the plurality of rating metrics satisfies a predetermined rating metric threshold.

16. A non-transitory computer-readable medium storing instructions that, when executed by at least one processor, cause a computing device to:
identify, from a processed biological representation a predicted biological relationship for an anchor compound or an anchor gene;
generate, from the predicted biological relationship for the anchor compound or the anchor gene, a plurality of digital text prompts, wherein the plurality of digital text prompts comprise the anchor compound or the anchor gene and a plurality of text rating instructions for a language machine learning model;
generate, from the plurality of digital text prompts utilizing the language machine learning model, a plurality of rating metrics according to the plurality of text rating instructions; and
combine the plurality of rating metrics to generate a program rating for the anchor compound or the anchor gene for initiating one or more compound exploration programs.

17. The non-transitory computer-readable medium of claim 16, further comprising instructions that, when executed by the at least one processor, cause the computing device to:
generate, utilizing a machine-learning model, a plurality of phenomic image embeddings from a plurality of perturbation images portraying a plurality of cell perturbations;
compare the plurality of phenomic image embeddings to determine a measure of similarity; and
identify the predicted biological relationship from the measure of similarity.

18. The non-transitory computer-readable medium of claim 17, further comprising instructions that, when executed by the at least one processor, cause the computing device to:
identify compound features corresponding to a compound and protein features corresponding to a protein;
generate, utilizing a compound protein-pocket interaction machine-learning model, a machine learning binding representation between the compound and the protein utilizing the compound features and the protein features; and
identify the predicted biological relationship from the machine learning binding representation.

19. The non-transitory computer-readable medium of claim 16, further comprising instructions that, when executed by the at least one processor, cause the computing device to:
identify a plurality of digital text prompt templates comprising one or more placeholder query fields; and
generate the plurality of digital text prompts by populating the one or more placeholder query fields of the plurality of digital text prompt templates based on the anchor compound or the anchor gene.

20. The non-transitory computer-readable medium of claim 16, further comprising instructions that, when executed by the at least one processor, cause the computing device to:
generate the plurality of digital text prompts further comprises generating, for the anchor compound or the anchor gene, a gene impact digital text prompt comprising gene impact text rating instructions; and
generate the plurality of rating metrics comprises generating, from the gene impact digital text prompt comprising the gene impact text rating instructions utilizing the language machine learning model, a gene impact rating metric indicating a measure of impact corresponding to the anchor gene.
